(19) United States
(12) Patent Application Publication
(10) Pub. No.: US 2014/0199309 A1
(43) Pub. Date: Jul. 17, 2014

(54) FRAGMENT OF SECRETED HEAT SHOCK PROTEIN-90ALPHA (HSP90ALPHA) AS VACCINES OR EPITOPE FOR MONOCLONAL ANTIBODY DRUGS OR TARGET FOR SMALL MOLECULE DRUGS AGAINST A RANGE OF SOLID HUMAN TUMORS

(71) Applicant: University of Southern California, Los Angeles, CA (US)

(72) Inventors: Wei Li, Altadena, CA (US); David T. Woodley, Altadena, CA (US); Mei Chen, Altadena, CA (US)

(73) Assignee: University of Southern California, Los Angeles, CA (US)

(21) Appl. No.: 14/225,311

(22) Filed: Mar. 25, 2014

Related U.S. Application Data
(63) Continuation of application No. 13/271,076, filed on Oct. 11, 2011, now abandoned.
(60) Provisional application No. 61/391,776, filed on Oct. 11, 2010.

(51) Int. Cl. C07K 14/435; C07K 16/18

(52) U.S. Cl. CPC C07K 14/435; C07K 16/18
USPC 424/135.1; 530/350; 530/324; 536/23.1; 530/387.9; 530/387.3; 424/139.1; 424/185.1; 514/19.3; 424/174.1; 514/44 R; 514/44 A; 424/155.1; 424/133.1; 435/252.33

(57) ABSTRACT

The invention provides methods for treating HIF-1α-overexpressing human tumors, inhibiting HIF-1α-overexpressing tumor invasion and preventing tumor metastasis, and/or promoting tumor prophylaxis, using various types of inhibitors against the Hsp90α from the tumors.

### CROSS REFERENCE TO RELATED APPLICATIONS

This application claims priority of U.S. Ser. No. 61/391,776 filed Oct. 11, 2010, the contents of which are herein incorporated by reference.

### GOVERNMENT LICENSE RIGHTS

The invention was made with government support under Grant Nos. RO1GM066193, RO1GM067100, GMAR67100-01, AR33625 and RO1AR46538 awarded by the National Institutes of Health. The government has certain rights in the invention.

### FIELD OF INVENTION

The invention relates to treatment of HIF-1α-overexpressing (i.e constitutive presence) tumors using inhibitors of Hsp90α or fragments thereof.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

In normal cells under normoxia (˜8% oxygen level in tissues), the hypoxia-inducible factor-1α (HIF-1α), a critical subunit of the master transcription factor for tissue oxygen homeostasis, is constantly synthesized and then immediately undergoes an O₂-dependent prolyl hydroxylation. This modification targets the HIF-1α protein to a ubiquitilation-proteasome machinery for degradation (23, 29). As a result, the overall steady-state level of HIF-1α is kept low, preventing HIF-1α from dimerization with HIF-1β. Under hypoxia, however, HIF-1α hydroxylation and subsequent degradation is suppressed, resulting in a rapid rise in HIF-1α levels in the cells. The increased HIF-1α proteins then dimerize with steady-state HIF-1β (ARNT) proteins to form the master transcriptional complex, HIF-1. HIF-1 in turn translocates into the nucleus and regulates expression of hypoxia response element (HRE)-containing genes in a p300/CBP-dependent manner (1).

In contrast, tissue hypoxia found in many solid tumors is caused by outgrowth of the rapidly proliferating tumor cells over the surrounding vascular network, which creates a distance that is longer than reach of oxygen supply from the nearest blood circulation (3, 29). Presumably, under constant ischemia, the tumor cells have undergone genetic changes to adapt alternative and self-supporting mechanisms for continued survival, expansion and progression, until neovascularization is built around them. Deregulated expression of HIF-1α is a well-characterized change in the tumor cells (23), which is attributed to action of activated oncogenes, inactivated tumor suppressor genes or deactivated enzymes for HIF-1α ubiquitylation and degradation (26). In animal models, the deregulated HIF-1α plays a crucial role in tumorigenesis. Down-regulation of HIF-1α expression or inhibition of the deregulated HIF-1α action slows tumor growth and renders the tumor more susceptible to killing by radio- and chemotherapies (28). In humans, the constitutively expressed HIF-1α is linked to large tumor size, high grade and lymph node-negative metastasis, which make the tumor less accessible to radiotherapy and chemotherapy (18). Therefore, the HIF-1α levels of expression in tumors have become a marker to predict possible outcomes of patients with tumor metastasis. While sabotaging the deregulated HIF-1α in tumor cells could in concept prevent tumor progression, directly targeting the intracellularly located HIF-1α or the enzymes that regulate HIF-1α stability proved to be challenging (26, 28).

The human Hsp90 chaperone family includes four confirmed members, the cytosolic Hsp90α and Hsp90β, the endoplasmic reticulum GRP74 and the mitochondrial TRAP1, encoded by distinct genes (6). Like HIF-1α in tumor cells, Hsp90α has also been found either quantitatively overexpressed or qualitatively overactivated in a variety of tumors (18). These “extra” or “overactive” Hsp90α proteins are thought to bind and protect the stability of oncogene products inside the cell (17, 25, 41). Such a seemingly higher degree of protection by Hsp90α in tumor cells than their proto-oncoprotein counterparts in surrounding normal cells has been taken as a strategy for developing anti-cancer drugs (42). Geldanamycin (GM, or benzoquinone ansamycin) and its derivatives, which bind and inhibit the ATP-binding and ATP hydrolysis functions of Hsp90, have been the focus of drug development for over a decade (25). GM itself proved to be too toxic even in animal models (36). A modified form of GM, 17-AAG (benzoquinone ansamycin 17-allylaminogeldanamycin), showed a promising efficacy at a dosage range with tolerable toxicity in pre-clinical studies and had entered several Phase 1 and phase 2/3 clinical trials since 1999 (33, 37). Several newer generations of chemically modified and less toxic GM-related drugs are being developed in ongoing clinical trials. However, the main hurdle remains as how to selectively target the oncogene-protecting activity of Hsp90 in tumors and spare the physiological function of Hsp90 in normal cells.

The statement of “1-2% of the total cellular proteins” has been widely used to describe the unusual abundance of Hsp90 protein inside most cell types. Taking 7000 proteins per cell, the content of Hsp90 protein would be 70-150 fold higher than any of the rest of the cellular proteins. Csermely and colleagues argued that, if chaperone were the only assigned function for Hsp90α by Mother Nature, such an over production of a single protein in cells would not be well tolerated by evolution (9). Therefore, they speculated that the major cellular function of Hsp90 is another yet recognized one that would require such an abundant storage of the protein. Studies of the past few years have discovered a surprising need for normal cells to secrete the “over stocked” Hsp90α for tissue repair or for tumor cells to constitutively secrete Hsp90α for invasion and metastasis (7, 38). More importantly, a key upstream regulator of Hsp90α secretion is hypoxia-induced HIF-1α (20, 42). In tumor cells, two groups used cell membrane-impermeable inhibitors of Hsp90α to specifically target secreted Hsp90α and showed the secreted Hsp90α was required for invasion of glioma, bladder cancer, breast cancer, prostate cancer, and melanoma in vitro and lung colonization by melanoma cells in vivo (39, 42). Since deregulated expression of HIF-1α is common in many solid tumors, the inventors hypothesized that in these tumors HIF-1α triggers Hsp90α secretion for migration, invasion and metastasis. In this study, the inventors have identified a novel 115-amino acid epitope, F-5, in secreted Hsp90α that might represent a new therapeutic target for HIF-1α-positive breast cancers and likely beyond.

### SUMMARY OF THE INVENTION

The present invention provides methods for treating HIF-1α-overexpressing cancer in a subject in need thereof using inhibitors of Hsp90α or fragments thereof. The methods comprise providing a composition comprising an inhibitor of Hsp90α or fragments thereof and administering a therapeutically effective amount of the composition to the subject so as to treat HIF-1α-overexpressing cancer in the subject.

The invention also provides methods for inhibiting HIF-1α-overexpressing cancer in a subject in need thereof using inhibitors of Hsp90α or fragments thereof. The methods comprise providing a composition comprising an inhibitor of Hsp90α or fragments thereof and administering a therapeutically effective amount of the composition to the subject so as to inhibit HIF-1α-overexpressing cancer in the subject.

The invention further provides methods for preventing metastasis of HIF-1α-overexpressing cancer in a subject in need thereof. The method comprises providing a composition comprising an inhibitor of Hsp90α or fragments thereof and administering a therapeutically effective amount of the composition to the subject so as to prevent metastasis of HIF-1α-overexpressing cancer in the subject.

Also provided herein are methods for promoting prophylaxis of HIF-1α-overexpressing cancer in a subject in need thereof. The method comprises providing a composition comprising an inhibitor of Hsp90α or fragments thereof and administering a therapeutically effective amount of the composition to the subject so as to promote prophylaxis of HIF-1α-overexpressing cancer in the subject.

The invention further provides methods for identifying inhibitors of Hsp90α. The methods comprise contacting the Hsp90α in a HSP90α positive cell or contacting LRP-1 in a LRP-1 positive cell with a molecule of interest, and determining whether the contact decreases or inhibits the binding of Hsp90α or fragments thereof to its receptor, a decrease or inhibition in binding being indicative that the molecule of interest is an inhibitor of Hsp90α or fragments thereof. Alternately, secretion of Hsp90α may also be assayed wherein reduction or inhibition of Hsp90α secretion in the presence of the molecule of interest indicates that the molecule of interest is an inhibitor of Hsp90α.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows that de-regulated HIF-1α is critical for breast cancer cell migration and invasion. (A) Western blot analysis of the HIF-1α levels in non-transformed breast epithelial cells, HBL-100, (panels a and b) and breast cancer cells, MDM-MB-231, (panels c and d) under either normoxia (21% O₂, lane 1) or hypoxia (1% O₂, lanes 2-5) over the indicated time points. Note: equal loadings of all samples and all procedures side-by-site. (B) The efficiency of FG-12 lentiviral infection in MDA-MB-231 cells, as indicated by expression of an in-cis CMV-driven GFP gene, followed by FACS analyses. The same field was shown with either phase contrast (left) or fluorescent lens (right). (C) Specific down-regulation of HIF-1α (panel a) or HIF-1β (panel d) proteins by FG-12-delivered shRNA, as indicated by Western blot analyses. (D) Twelve-well tissue culture plates were pre-coated with type I collagen (20 μg/ml, 2 hr). Serum-starved cells were plated (250,000 cells/well) in serum-free medium and >90% fo the cells attached within 2 hours. The wound closure was photographed and quantified as Average Gap (AG) (40), (n=3, p<0.05). (E) Down-regulation of HIF-1α or HIF-1β inhibited MDA-MB-231 cell invasion through a Matrigel barrier (panels b and c vs. panel a), according to manufacturer's protocol. Note: O.D. reading (Bio-Rad Protein Assay at 590 nm) on the penetrated cells only. The data are expressed as means±s. d. (n=4, p<0.05)

FIG. 2 shows that de-regulated HIF-1α uses secreted Hsp90α for migration and invasion. (A) Serum-free conditioned medium (CM) (25 μl of 10× concentrated) of HBL-100 (lanes 1 and 2) or MDA-MB-231 (lanes 3 and 4) cells incubated under normoxia (N) or hypoxia (H) for 14 hours was analyzed for the presence of Hsp90α proteins by Western. (B) CM of HIF-1α- or HIF-1β-downregulated cells was analyzed for the presence of Hsp90α (a, lanes 2, 3 vs. lane 1). 1.0 ml of 1×CM was concentrated 20 times and subjected to zymography gel analysis (Methods) (b, lanes 1-3). (C) Re-introduction of wt and CA mutant (a, lanes 2, 3), but not DN mutant (b, lanes 4), of HIF-1α genes rescued Hsp90α secretion in HIF-1α downregulated MDA-MB-231 cells (c, lanes 2 and 3 vs. lanes 1 and 4). (D) Colloidal gold migration assays show that anti-Hsp90α neutralizing antibodies blocked individual MDA-MB-231 cell motility in a dose-dependent manner (panels c, d, e vs. a, b). The addition of excess amount of recombinant Hsp90α reversed the inhibition by the antibodies (panel f). MI, migration index (%) (40). (E) Anti-Hsp90α neutralizing antibodies blocked MDA-MB-231 cell invasion through a Matrigel in a dose-dependent manner (panels c, d, e vs. a, b). The addition of excess amount of recombinant Hsp90α reversed the inhibition of invasion by the antibodies (panel f).

FIG. 3 shows the comparison of cellular Hsp90α storage in normal versus tumor cells. The percentage (%) of Hsp90α protein in reference to the total cellular proteins in various cell types indicated was estimated (see text). A) Anti-Hsp90α antibody blots (Western) of four normal cell types versus recombinant (recom.) Hsp90α proteins and B) Western blots of four tumor cells versus recombinant Hsp90α proteins. Note: The intensities of the bands within a given cell type with recombinant Hsp90α, but not between different cell types, are results of identical conditions (including film exposure time) and therefore comparable. The readings of densitometry scanning of the Hsp90α bands from the cell lysates were converted to μg of protein according to a standard curve made from the readings of the known μgs of recombinant Hsp90α loaded side-by-side. Finally, Hsp90α (μg) in a given volume of the lysate/total proteins (μg) in the same volume of the lysate ×100=% of Hsp90α in the given cell type. Quantitation of the results from multiple pairs of lysate versus recombinant Hsp90α and results of three independent experiments was shown in Table 1.

FIG. 4 shows that F-5 epitope in secreted Hsp90α mediates tumor cell migration and invasion. (A) A summary of truncated peptides of Hsp90α that still retain full or partial pro-motility activity of full-length Hsp90α. Cell motility data is summary (%) of colloidal gold motility and “scratch” assays combined (n=3, each assay, p<0.05). (B) FPLC-purified full-length, F-2, F-5 and F-7 were visualized in SDS gel stained with Coomassie Brilliant Blue (lanes 1 to 4), with indicated amounts of BSA as controls (lanes 5 to 7). F-9 is a synthetic peptide. (C) The five peptides with their optimized concentrations were tested for rescuing invasion defect of HIF-1α-downregulated MDA-MB-231 cells. D). Quantitation of the invasion data (n=3, p<0.05). (E) Lysates of MDA-MB-231 cells infected with vector alone (lane 1) or lentivirus carrying shRNA against LRP-1 receptor (lane 2) were analyzed by Western with anti-LRP-1 antibody. (F) LRP-1-downregulated cells were unable to invade as the control cells (panel b vs. a) and Hsp90α was unable to rescue the invasion defect of the LRP-1-downregulated cells (n=4, p<0.05).

FIG. 5 shows that Hsp90α signaling is essential for MDA-MB-231 cell lung colonization and tumor formation in vivo. (A) Lentiviral system-, FG-12, mediated shRNA-LRP-1 delivery and down-regulation of endogenous LRP-1 in MDAMB-231 cells (lane 2 vs. lane 1). (B) Western blot screening of normal and cancer cell lines for expression of LRP-1 receptor. (C) Constitutive expression of HIF-1α in MDA-MB-468 cells under either normoxia (N) or hypoxia (H) (panel a). Constitutive secretion of Hsp90α by MDA-MB-468 cells (panel c). (D) 1×106 of luciferase-engineered MDAMB-231 cells infected with either vector only (a) or vector carrying shRNA-LRP-1 (b) or LRP-1−/− MDAMB-468 cells (c) were injected into the tail vein of SCID mice (n=7 per group). Whole body bioluminescence imaging of the mice was performed once a week. Representative images (2 per group) of the mice show lung colonization of the injected cells at day of 1, 14, 28, 56 and 70. (E) Between day 70 to 75 (4-5 of the 7 mice injected with the control MDAMB-231 cells died of tumors.), mice were put into sleep and the entire lung removed and sectioned for H&E staining analyses. Blue and green arrows indicate invading tumor nodule and normal lung tissue, respectively. T, tumor nodule; L, lung parenchyma; scale bars as indicated. (F) One-way analysis of variance analysis (ANOVA) of the staining shown in FIG. 5E was carried out to compare the number of tumors in the lungs of mice carrying MDA-MB-231-vector, MDA-MB-231-LRP-1RNAi and MDA-MB-468 cells. * p<0.05, ** p<0.003

FIG. 6 shows a model of secreted Hsp90α as a potential target for HIF-1α-positive cancers. The severe hypoxia often found at the center of a tumor causes constitutive accumulation of HIF-1α. The deregulated HIF-1α triggers secretion of Hsp90α via exosomes. The secreted Hsp90α binds, via F-5 epitope, to cell surface LRP-1 receptor and promotes motility and invasion of tumor cells in an autocrine fashion. While current clinical trials focus on intracellular HIF-1α, the Applicants propose that targeting the F-5 epitope of secreted Hsp90α is more effective and safer in treatment of cancer patients.

FIG. 7 shows that wt-HIF-1α and CA-HIF-1α were able to rescue the defect of cell motility of the endogenous HIF-1α-depleted cells. Twelve-well tissue culture plates were uncoated or coated with type I collagen (40 μg/ml, 2 hr). Unattached collagens were removed by washing with HBSS buffer. Serum-starved (18 h) MDA-MB-231 were plated (250,000 cells/well). In serum-free medium, so that the cell density reached 90% confluence within 2 hours. The “wounds” were made with a p-200 pipette tip. The wound closure was photographed and quantitation (Average Gap, AG) carried out as described (Li et al., 2004, MBC, 15:294-309)). These results shown here are reproducible in three independent experiments (n=3, p<0.05).

FIG. 8 shows that treatment of cells with DMA, a specific inhibitor of protein secretion blocked Hsp90α secretion. (A) ˜5×10⁶ MDA-MB-231 cells were cultured in three 10 cm dishes with 4 ml of serum-free medium. 15 μl of PBS or PBS containing BFA (30 nM) or DMA (15 nM) were added to the medium. Following 12-hour incubation, the media were collected, concentrated 10× and subjected to Western blot analysis with anti-Hsp90α antibodies. (B) The MDA-MB-231 cells were subjected to invasion assays in the absence (a and b) or presence of BFA (c) or DMA (d, e, f). DMA Inhibited invasion in a concentration-dependent manner (n=3, p=0.044, ±s.d.).

FIG. 9 shows that F-5 rescued the invasion defect of the cells. The F-5 peptide was tested for its ability to rescue the invasion defect of the endogenous HIF-1β-downregulated MDA-MB-231 cells. Panels: (a) Lac-Z-RNAi-infected cells; (b) HIF-1β-RNAi-infected cells with added BSA; (c to e) increasing amount of F-5 peptide added to HIF-1β-RNAi-infected cells. Quantitation of the invasion data shown in O.D. (n=4, p<0.05).

FIG. 10 shows that in both migration and invasion assays, Hsp70, gp96 and CRT showed limited effects. (A) The indicated recombinant proteins with their optimized concentrations were tested for their ability to promote human keratinocyte migration (that do not secreted Hsp90 under physiological conditions.) (n=4, * p<0.05). (B) Rescue the invasion defect of endogenous HIF-1α-downregulated MDA-MB-231 cells by the indicated peptides/proteins. One representative experiment of two invasion rescue assays (n=2). * p<0.05 was based on three wells, per condition, per experiment, using “-” as the baseline.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Conditions” and “disease conditions,” as used herein may include, but are in no way limited to any form of cancer.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Cancer” and “cancerous” refer to or describe the physiological condition in mammals that is typically characterized by unregulated cell growth. Examples of cancer include, but are not limited to, brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Tumor,” as used herein refers to all neoplastic cell growth and proliferation, whether malignant or benign, and all pre-cancerous and cancerous cells and tissues.

“HIF-1” as used herein refers to hypoxia-inducible factor-1. HIF-1α-overexpressing cancer refers to cancers in which HIF-1α is overexpressed in tumor cells. The majority of common human cancers in at least 15 organs over express HIF-1α (see the complete list by G. L. Semenza (2007) Drug Discovery Today, Vol. 12, Page 853-859)

“LRP-1” or “LRP1” as used herein refers to aow density lipoprotein receptor-related protein 1, also known as alpha-2-macroglobulin receptor (A2MR), apolipoprotein E receptor (APOER) or cluster of differentiation 91 (CD91). LRP-1 is a receptor for Hsp90α.

HSP90α or Hsp90α as used herein refers to the heat shock protein 90α.

“Isolated Hsp90α”, “purified Hsp90α”, “isolated fragments of Hsp90α” or “purified fragments of Hsp90α” as used herein refer to Hsp90α proteins or fragments thereof that are expressed and removed from non-Hsp90α or fragments thereof and/or removed from cellular constituent that are associated with or impair the activity of Hsp90α or fragments there.

Deregulated overexpression of hypoxia-inducible factor-1alpha (HIF-1α) is a hallmark of many solid tumors. However, directly targeting HIF-1α proved to be challenging. Applicants have previously shown that secreted heat shock protein-90alpha (Hsp90α) is a critical downstream effector of HIF-1α, raising an exciting possibility that targeting the action of secreted Hsp90α of HIF-1α-overexpressing tumors is easier and more specific. Applicants have tested this possibility in the HIF-1α-overexpressing and metastatic breast cancer cells, MDA-MB-231. Down-regulation of HIF-1α blocked Hsp90α secretion and invasiveness of the cells. Re-introducing an active, but not inactive, HIF-1α gene into endogenous HIF-1α-depleted MDA-MB-231 cells rescued Hsp90α secretion and invasion. Direct inhibition of Hsp90α secretion, neutralization of the secreted Hsp90α function or removal of the cell surface LRP-1 receptor for secreted Hsp90α all dramatically decreased the cell invasion in vitro and lung colonization and tumor formation in nude mice. Most importantly, Applicants have located the key epitope in secreted Hsp90α, called F-5. Supplementation with F-5 peptide alone was sufficient to bypass the blockade of HIF-1α depletion in MDA-MB-231 cells and resumed invasion. Since normal cells secrete Hsp90α only under a stress, targeting the F-5 epitope should be more effective and less toxic in treatments of HIF-1α-positive tumors in humans.

### Nucleic Acids and Proteins of the Invention

The invention provides nucleic acid sequences of Hsp90α which may be targeted to treat HIF-1α-overexpressing cancer, inhibit HIF-1α-overexpressing cancer, prevent metastasis of HIF-1α-overexpressing cancer and/or promote prophylaxis of HIF-1α-overexpressing cancer. The nucleic acid sequences provided herein may also be targeted to mitigate the effects of HIF-1α-overexpressing cancers, to reduce the severity of HIF-1α-overexpressing cancers, to reduce the likelihood of developing HIF-1α overexpressing cancers and/or to slow the progression of HIF-1α-overexpressing cancers. The nucleic acid molecules can be DNA or RNA. In one embodiment, the DNA sequence of full-length Hsp90α is an isolated cDNA sequence, as shown in SEQ ID NO: 1. In a further embodiment, the cDNA sequence encoding Hsp90α is set forth in SEQ ID NO: 1, starting at adenosine at position 1 and ending at adenosine at position 2199.

The invention also provides the nucleic acid sequences of fragments of Hsp90α which may be targeted to treat HIF-1α-overexpressing cancer, inhibit HIF-1α-overexpressing cancer, prevent metastasis of HIF-1α-overexpressing cancer and/or promote prophylaxis of HIF-1α-overexpressing cancer. The nucleic acid sequences provided herein may also be targeted to mitigate the effects of HIF-1α-overexpressing cancers, to reduce the severity of HIF-1α-overexpressing cancers, to reduce the likelihood of developing HIF-1α overexpressing cancers and/or to slow the progression of HIF-1α-overexpressing cancers. The nucleic acid molecules can be DNA or RNA. In one embodiment, the DNA sequence of the fragment of Hsp90α is an isolated cDNA sequence as shown in SEQ ID NO: 3. In another embodiment, the DNA sequence of the fragment of Hsp90α is an isolated cDNA sequence as shown in SEQ ID NO: 5.

The nucleic acid molecules herein enable the isolation of Hsp90α homologs, alternatively spliced isoforms, allelic variants and mutant forms of Hsp90α protein as well as their coding and gene sequences. In one embodiment, the source of Hsp90α homologs is mammalian organisms. Homologues and naturally occurring allelic variants of human Hsp90α or fragments thereof described herein may share a high degree of homology with Hsp90α of other mammals, yeast, bacteria or plants. Such sequences contain at least 70% homology, preferably at least 80% homology, most preferably at least 90% homology to the human Hsp90α or fragments thereof as described in SEQ ID NOs: 1, 3 and/or 5.

Vectors encoding Hsp90α or fragments thereof having sequences set forth in any one or more of SEQ ID NOs: 1, 3 and/or 5 are also provided herein.

The invention further provides the amino acid sequences of Hsp90α and fragments thereof which may be targeted to treat HIF-1α-overexpressing cancer, inhibit HIF-1α-overexpressing cancer and/or prevent metastasis of HIF-1α-overexpressing cancer. In one embodiment, the amino acid sequence of full length Hsp90α is set forth in SEQ ID NOs: 1 and 2. In another embodiment, the amino acid sequence encoding a fragment of Hsp90α is set forth in SEQ ID NOs: 3 and 4. In a further embodiment, the DNA sequence encoding a fragment of Hsp90α is set forth in SEQ ID NOs: 5 and 6. These sequences may be targeted, for example, for mutagenesis so as to alter the sequence and thereby the function of Hsp90α or fragments thereof.

Also provided herein are Hsp90α proteins or fragments thereof which may be targeted to treat HIF-1α-overexpressing cancer, inhibit HIF-1α-overexpressing cancer, prevent metastasis of HIF-1α-overexpressing cancer and/or promote prophylaxis of HIF-1α-overexpressing cancer. The Hsp90α proteins or fragments thereof provided herein may also be targeted to mitigate the effects of HIF-1α-overexpressing cancers, to reduce the severity of HIF-1α-overexpressing cancers, to reduce the likelihood of developing HIF-1α overexpressing cancers and/or to slow the progression of HIF-1α-overexpressing cancers. In one embodiment, the Hsp90α protein comprises 732 amino acids as shown in SEQ ID NOs: 1 and 2, beginning with methionine at position 1 and ending with Aspartic acid at position 732. In another embodiment, the fragment of Hsp90α comprises 115 amino acids as shown in SEQ ID NOs: 3 and 4, beginning with Glutamic acid at position 1 and Aspartic acid at position 115. In an additional embodiment, the fragment of Hsp90α comprises 27 amino acids as shown in SEQ ID NOs: 5 and 6, beginning with Serine at position 1 and Glutamic acid at position 27.

The invention also provides mutants of Hsp90α or mutants of fragments of Hsp90α. Mutations in Hsp90α or fragments thereof may result from insertion, deletion, frameshift, knockout and/or substitution mutations. Mutant form of Hsp90α or fragments thereof may treat HIF-1α-overexpressing cancer, inhibit HIF-1α-overexpressing cancer, prevent metastasis of HIF-1α-overexpressing cancer, promote prophylaxis of HIF-1 positive cancer, mitigate the effects of HIF-1α-overexpressing cancers, reduce the severity of HIF-1α-overexpressing cancers, reduce the likelihood of developing HIF-1α overexpressing cancers and/or slow the progression of HIF-1α-overexpressing cancers.

Hsp90α proteins and/or fragments thereof, such as those encoded by any one or more of SEQ ID NOs: 1-6, may be in an isolated form. These proteins may be expressed in vitro or in vivo. A variety of methods may be used to isolate and purify Hsp90α proteins or fragments thereof and would be easily known to a person of ordinary skill in the art.

The invention also provides methods for producing Hsp90α and/or fragments thereof. In one embodiment, Hsp90α or fragments thereof are cloned into a vector which is compatible with a host cell. The host cell may be a prokaryotic cell or a eukaryotic cell. The method involves growing the host-vector system transfected with a vector encoding Hsp90α, derivatives or fragments thereof, so as to produce the Hsp90α molecules, derivatives or fragments thereof, in the host and then recovering the Hsp90α molecules, derivatives or fragments thereof.

### Therapeutic Methods of the Invention

The present invention describes methods and kits for using Hsp90α inhibitors to treat HIF-1α-overexpressing cancer, inhibit HIF-1α-overexpressing cancer and/or prevent metastasis of HIF-1α-overexpressing cancer While not wishing to be bound by any particular theory, the inventors believe that their findings that Hsp90α inhibitors, especially those which target the sequences set forth in SEQ ID NOs: 3, 4, 5 and/or 6, inhibit cancer cell migration, invasion and progression, supporting the notion that administration of these compounds in a clinical setting may result in the treatment, inhibition, metastasis-prevention and/or prophylaxis-promotion of HIF-1α-overexpressing cancers.

The instant invention provides methods for treating HIF-1α-overexpressing cancer in a subject in need thereof using inhibitors of Hsp90α or fragments thereof. The methods comprise providing a composition comprising an inhibitor of Hsp90α and administering a therapeutically effective amount of the composition to the subject so as to treat HIF-1α-overexpressing cancer in the subject. The composition may further comprise other pharmaceuticals, including but not limited to cancer vaccines and/or other chemotherapeutic agents. In one embodiment, the inhibitor targets secreted Hsp90α. In another embodiment, the inhibitor prevents the secretion of Hsp90α. In a further embodiment, the inhibitor targets full length Hsp90α as set forth in SEQ ID NOs: 1 and/or 2. In a preferred embodiment, the inhibitor targets the sequence set forth in SEQ ID NOs: 3, 4, 5 or 6. In an additional embodiment, the Hsp90α inhibitor is a derivative of Hsp90α and/or a derivative of a fragment thereof.

The invention also provides methods for inhibiting HIF-1α-overexpressing cancer in a subject in need thereof using inhibitors of Hsp90α. The methods comprise providing a composition comprising an inhibitor of Hsp90α and administering a therapeutically effective amount of the composition to the subject so as to inhibit HIF-1α-overexpressing cancer in the subject. The composition may further comprise other pharmaceuticals, including but not limited to cancer vaccines and/or other chemotherapeutic agents. In one embodiment, the inhibitor targets secreted Hsp90α. In another embodiment, the inhibitor prevents the secretion of Hsp90α. In a further embodiment, the inhibitor targets full length Hsp90α as set forth in SEQ ID NOs: 1 and/or 2. In a preferred embodiment, the inhibitor targets the sequence set forth in SEQ ID NOs: 3, 4, 5 or 6. In an additional embodiment, the Hsp90α inhibitor is a derivative of Hsp90α and/or a derivative of a fragment thereof.

The invention further provides methods for preventing metastasis of HIF-1α-overexpressing cancer in a subject in need thereof. The method comprises providing a composition comprising an inhibitor of Hsp90α and administering a therapeutically effective amount of the composition to the subject so as to prevent metastasis of HIF-1α-overexpressing cancer in the subject. The composition may further comprise other pharmaceuticals, including but not limited to cancer vaccines and/or other chemotherapeutic agents. In one embodiment, the inhibitor targets secreted Hsp90α. In another embodiment, the inhibitor prevents the secretion of Hsp90α. In a further embodiment, the inhibitor targets full length Hsp90α as set forth in SEQ ID NOs: 1 and/or 2. In a preferred embodiment, the inhibitor targets the sequence set forth in SEQ ID NOs: 3, 4, 5 or 6. In an additional embodiment, the Hsp90α inhibitor is a derivative of Hsp90α and/or a derivative of a fragment thereof.

Also provided herein are methods for promoting prophylaxis of HIF-1α-overexpressing cancer in a subject in need thereof. The method comprises providing a composition comprising an inhibitor of Hsp90α and administering a therapeutically effective amount of the composition to the subject so as to promote prophylaxis of HIF-1α-overexpressing cancer in the subject. The composition may further comprise other pharmaceuticals, including but not limited to cancer vaccines and/or other chemotherapeutic agents. In one embodiment, the inhibitor targets secreted Hsp90α. In another embodiment, the inhibitor prevents the secretion of Hsp90α. In a further embodiment, the inhibitor targets full length Hsp90α as set forth in SEQ ID NOs: 1 and/or 2. In a preferred embodiment, the inhibitor targets the sequence set forth in SEQ ID NOs: 3, 4, 5 or 6. In an additional embodiment, the Hsp90α inhibitor is a derivative of Hsp90α and/or a derivative of a fragment thereof.

The invention also provides methods for mitigating the effects of HIF-1α-overexpressing cancers, reducing the severity of HIF-1α-overexpressing cancers, reducing the likelihood of developing HIF-1α overexpressing cancers and/or slowing the progression of HIF-1α-overexpressing cancers. The methods comprise providing a composition comprising an inhibitor of Hsp90α and administering a therapeutically effective amount of the composition to the subject so as to mitigate the effects of HIF-1α-overexpressing cancers, reduce the severity of HIF-1α-overexpressing cancers, reduce the likelihood of developing HIF-1α overexpressing cancers and/or slow the progression of HIF-1α-overexpressing cancers in the subject. The composition may further comprise other pharmaceuticals, including but not limited to cancer vaccines and/or other chemotherapeutic agents. In one embodiment, the inhibitor targets secreted Hsp90α. In another embodiment, the inhibitor prevents the secretion of Hsp90α. In a further embodiment, the inhibitor targets full length Hsp90α as set forth in SEQ ID NOs: 1 and/or 2. In a preferred embodiment, the inhibitor targets the sequence set forth in SEQ ID NOs: 3, 4, 5 or 6. In an additional embodiment, the Hsp90α inhibitor is a derivative of Hsp90α and/or a derivative of a fragment thereof.

The Hsp90α inhibitor may target Hsp90α as set forth in SEQ ID NOs: 1 and/or 2. Hsp90α may be secreted or non-secreted Hsp90α. In a preferred embodiment, the inhibitor of Hsp90α targets the amino acids of Hsp90α as set forth in SEQ ID NOs: 3, 4, 5 or 6. In a further embodiment, the Hsp90α inhibitor targets the receptor of Hsp90α, LRP-1, such that Hsp90α and/or fragments thereof and/or derivatives thereof cannot bind LRP-1, or may exhibit reduced or no binding. Hsp90α may also bind to other targets, including MMP2 (matrix matelloproteinase-2) and receptor tyrosine kinases. The Hsp90α inhibitors may also target MMP2 and receptor tyrosine kinases.

In another embodiment, Hsp90α inhibitors are selected from the group consisting of a small molecule, a peptide, an antibody or a fragment thereof and a nucleic acid molecule. The nucleic acid molecules that inhibit Hsp90α may be, for example, siRNA molecules. The antibodies which inhibit Hsp90α may be any one or more of monoclonal antibodies or fragments thereof, polyclonal antibodies or a fragments thereof, chimeric antibodies, humanized antibodies, human antibodies, or single chain antibodies. According to certain aspects, the Hsp90α inhibitor may be used in combination with other pharmaceuticals, including but not limited to cancer vaccines and/or other chemotherapeutic agents.

In a further embodiment the inhibitors of Hsp90α inhibit the receptor of Hsp90α and are selected from the group consisting of small molecules, peptides, antibodies or fragments thereof and nucleic acid molecules. The nucleic acid molecules that inhibit the Hsp90α receptor may be, for example, siRNA molecules. The antibodies which inhibit the Hsp90α receptor may be any one or more of monoclonal antibodies or fragments thereof, polyclonal antibodies or a fragments thereof, chimeric antibodies, humanized antibodies, human antibodies, or single chain antibodies. According to certain aspects, the Hsp90α inhibitor may be used in combination with other pharmaceuticals, including but not limited to cancer vaccines and/or other chemotherapeutic agents.

Additional embodiments of the instant invention provide mutations in Hsp90α which result in Hsp90α inhibition. Mutations in Hsp90α may result in prevention/inhibition of Hsp90α secretion. Alternately, mutations in Hsp90α may, for example, render secreted Hsp90α unstable or unable to bind its receptor, LRP1. Further embodiments provide mutations in the Hsp90α receptor LRP1 such that LRP1 can no longer bind Hsp90α thus inhibiting Hsp90α. Mutations in Hsp90α or its receptor which render Hsp90α inactive and/or inhibited include but are not limited to insertions, deletions, frameshifts, knockouts and/or substitutions.

The invention further provides methods for treating, inhibiting, preventing metastasis of and/or promoting prophylaxis of HIF-1α-overexpressing cancer in a subject in need thereof using sensitized antigen presenting cells. The method comprises providing a composition comprising sensitized antigen presenting cells, wherein the antigen presenting cells are sensitized with any one or more of the peptides having the amino acid sequence set forth in SEQ ID NO:3, SEQ ID NO:4, SEQ ID NO: 5 and/or SEQ ID NO:6 and administering a therapeutically effective amount of the composition to the subject so as to treat, inhibit, prevent metastasis of and/or promote prophylaxis of HIF-1α-overexpressing cancer in the subject. In one embodiment, examples of antigen presenting cells include dendritic cells, macrophages and B cells. In another embodiment, examples of antigen presenting cells include fibroblast, thymic epithelial cells, thyroid epithelial cells, glial cells, pancreatic beta cells and vascular endothelial cells, all of which may be costimulated by cytokines such as interferon gamma (IFNγ). According to certain aspects, the Hsp90α inhibitor may be used in combination with other pharmaceuticals, including but not limited to cancer vaccines and/or other chemotherapeutic agents.

The invention further provides methods for treating, inhibiting, preventing metastasis of and/or promoting prophylaxis of HIF-1α-overexpressing cancer in a subject in need thereof using activated T cells. The methods comprise providing a composition comprising activated T cells, wherein the activated T cells are activated with any one or more of the peptides having the amino acid sequence set forth in SEQ ID NO:3, SEQ ID NO:4, SEQ ID NO: 5 and/or SEQ ID NO:6 and administering a therapeutically effective amount of the composition to the subject so as to treat, inhibit, prevent metastasis of and/or promote prophylaxis of HIF-1-positive cancer in the subject. In one embodiment, the activated T cells are CD8+T cells.

The invention also provides methods for mitigating the effects of HIF-1α-overexpressing cancers, reducing the severity of HIF-1α-overexpressing cancers, reducing the likelihood of developing HIF-1α-overexpressing cancers and/or slowing the progression of HIF-1α-overexpressing cancers. The methods comprise providing a composition comprising activated T cells, wherein the activated T cells are activated with any one or more of the peptides having the amino acid sequence set forth in SEQ ID NO:3, SEQ ID NO:4, SEQ ID NO: 5 and/or SEQ ID NO:6 and administering a therapeutically effective amount of the composition to the subject so as to mitigate the effects of HIF-1α-overexpressing cancers, reduce the severity of HIF-1α-overexpressing cancers, reduce the likelihood of developing HIF-1α overexpressing cancers and/or slow the progression of HIF-1α-overexpressing cancers in the subject.

The Hsp90α inhibitors and the Hsp90α receptor inhibitors may be administered intravenously, intramuscularly, intraperitonealy, orally or via inhalation.

The subjects in the claimed invention may be any one or more of human, non-human primate, monkey, ape, dog, cat, cow, horse, rabbit, mouse and rat.

### Dosages of the Invention

In some embodiments of the invention, the effective amounts of Hsp90α inhibitor in the composition can be in the range of about 1-5 mg/day, 5-10 mg/day, 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day. In a preferred embodiment, the inhibitor at the aforementioned dosages targets the sequence set forth in SEQ ID NOs: 3, 4, 5 or 6. In an additional embodiment, the Hsp90α inhibitor is a derivative of Hsp90α and/or a derivative of a fragment thereof and is administered at any of the above dosages. The inhibitor of Hsp90α may target secreted Hsp90α or non-secreted Hsp90α.

In further embodiments of the invention, the effective amount of Hsp90α inhibitor for use with the claimed methods may be in the range of 1-5 mg/kg, 5-10 mg/kg, 10-50 mg/kg, 50-100 mg/kg, 100-150 mg/kg, 150-200 mg/kg, 100-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg or 2900-3000 mg/kg. In a preferred embodiment, the inhibitor at the aforementioned dosages targets the sequence set forth in SEQ ID NOs: 3, 4, 5 or 6. In an additional embodiment, the Hsp90α inhibitor is a derivative of Hsp90α and/or a derivative of a fragment thereof and is administered at any of the above dosages. The inhibitor of Hsp90α may target secreted Hsp90α or non-secreted Hsp90α.

In additional embodiments of the invention, the effective amounts of Hsp90α receptor inhibitor in the composition can be in the range of about 1-5 mg/day, 5-10 mg/day, 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day.

In other embodiments, the effective amount of Hsp90α inhibitor for use with the claimed methods may be in the range of 1-5 mg/kg, 5-10 mg/kg, 10-50 mg/kg, 50-100 mg/kg, 100-150 mg/kg, 150-200 mg/kg, 100-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg or 2900-3000 mg/kg.

Typical dosages of an effective amount of Hsp90α inhibitor can be in the ranges recommended by the manufacturer where known therapeutic compounds are used, and also as indicated to the skilled artisan by the in vitro responses or responses in animal models. The same or similar dosing can be used in accordance with various embodiments of the present invention, or an alternate dosage may be used in connection with alternate embodiments of the invention. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models.

### Screening Methods of the Invention

Another aspect of the invention relates to assays and methods for identifying compounds that inhibit Hsp90α and/or Hsp90α receptor. In one embodiment, the method comprises contacting the Hsp90α in a Hsp90α positive cell with a molecule of interest and determining whether the contact decreases or inhibits the binding of Hsp90α to its receptor. A decrease or inhibition in the binding of Hsp90α to Hsp90α receptor indicates that the molecule of interest is an inhibitor of Hsp90α. In a further embodiment, the method comprises separately contacting each of a plurality of samples to be tested, wherein the plurality of samples may be, for example, more than about 10⁴ samples or more than about 5×10⁴ samples. The inhibitor may target Hsp90α as set forth in SEQ ID NOs: 1 or 2. Preferably, the inhibitor targets the sequence set forth in SEQ ID NOs: 3, 4, 5 or 6. Additionally, the Hsp90α inhibitor is a derivative (for example, a mutant) of Hsp90α and/or a derivative of a fragment thereof.

In another embodiment, the method for identifying inhibitors of Hsp90α comprise contacting LRP-1 in a LPR-1 positive cells with a molecule of interest and determining whether the contact decreases or inhibits the binding of Hsp90α to LRP-1. A decrease or inhibition in binding of Hsp90α to LRP-1 indicates that the molecule of interest is an inhibitor of Hsp90α. In a further embodiment, the method comprises separately contacting each of a plurality of samples to be tested, wherein the plurality of samples may be, for example, more than about 10⁴ samples or more than about 5×10⁴ samples. In an embodiment, the inhibitor is a derivative (for example, a mutant) of LRP-1 such that Hsp90α either cannot bind LRP-1 or exhibit reduced binding to LRP-1.

In a further embodiment, the method of identifying inhibitors of Hsp90α comprises contacting the Hsp90α in a Hsp90α positive cell with a molecule of interest and determining whether the contact decreases or inhibits the secretion of Hsp90α. A decrease or inhibition in the secretion of Hsp90α indicates that the molecule of interest is an inhibitor of Hsp90α. In a further embodiment, the method comprises separately contacting each of a plurality of samples to be tested, wherein the plurality of samples may be, for example, more than about 10⁴ samples or more than about 5×10⁴ samples. The inhibitor may target Hsp90α as set forth in SEQ ID NOs: 1 or 2. Preferably, the inhibitor targets the sequence set forth in SEQ ID NOs: 3, 4, 5 or 6. Additionally, the Hsp90α inhibitor is a derivative (for example, a mutant) of Hsp90α and/or a derivative of a fragment thereof.

The compound of interest that inhibits Hsp90α and/or LRP-1 may be any one or more of a small molecule, a peptide, an antibody or a fragment thereof and a nucleic acid molecule.

Assays that may be employed to identify compounds that inhibit Hsp90α and/or fragments thereof include but are not limited to microarray assay, quantitative PCR, Northern blot assay, Southern blot assay, Western blot assay immunohistochemical assays, binding assays, gel retardation assays or assays using yeast two-hybrid systems. A person skilled in the art can readily employ numerous techniques known in the art to determine whether a particular agent inhibits Hsp90α and/or fragments thereof.

### Pharmaceutical Compositions

In various embodiments, the present invention provides pharmaceutical compositions including a pharmaceutically acceptable excipient along with a therapeutically effective amount of Hsp90α inhibitor. The inhibitor may target Hsp90α as set forth in SEQ ID NOs: 1 or 2. Preferably, the inhibitor targets the sequence set forth in SEQ ID NOs: 3, 4, 5 or 6. Additionally, the Hsp90α inhibitor is a derivative (for example, a mutant) of Hsp90α and/or a derivative of a fragment thereof.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use.

Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Kits of the Invention

The present invention is also directed to kits to treat HIF-1α-overexpressing cancer, inhibit HIF-1α-overexpressing cancer, prevent metastasis of HIF-1α-overexpressing cancer and/or promote prophylaxis of HIF-1α-overexpressing cancer. Also provided herein are kits for mitigating the effects of HIF-1α-overexpressing cancers, reducing the severity of HIF-1α-overexpressing cancers, reducing the likelihood of developing HIF-1α-overexpressing cancers and/or slowing the progression of HIF-1α-overexpressing cancers. The kit is an assemblage of materials or components, including at least one of the inventive compositions. Thus, in some embodiments the kit contains a composition including Hsp90α inhibitor, as described above.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or prevent treat HIF-1α-overexpressing cancer, inhibit HIF-1α-overexpressing cancer, prevent metastasis of HIF-1α-overexpressing cancer and/or promote prophylaxis of HIF-1α-overexpressing cancer in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing a inhibitor of Hsp90α or fragments thereof. The packaging material generally has an external label which indicates the contents and/or purpose of the kit and/or its components.

### EXAMPLES

The following example is provided to better illustrate the claimed invention and is not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Experimental Methods

Cell lines screened for deregulated HIF-1α expression included: HB1-100 human breast epithelial cell line, four human breast cancer cell line (MDA-MB-231, MDA-MB-468, MDA-MB-435 and MCF-7), M24 and M21 human melanoma cell lines, U251 and U87 human glioma cell lines, A172 human glioblastoma cell line, PC3 human prostate cancer cell line and A431 human skin carcinoma cell line. Native rat-tail type I collagen was from BD Biosciences (Bedford, Mass.). Colloidal gold (gold chloride, G4022) was purchased from SIGMA (St. Louis, Mich.). The cDNAs that encode HIF-1α (wt), HIF-1αCA5 (constitutively active) and HIF-1αΔNBΔAB (dominant negative) were gifts from Dr. Gregg Semenza (Johns Hopkins University) and cloned into lentiviral vector, pPPLsin.MCS-Deco (20). ShRNAs against human HIF-1α and HIF-1β were cloned in lentiviral FG-12 system, as previously described (39). Anti-HIF-1α antibody (#610958) and anti-HIF-1β antibody (#611078) were from BD Transduction Laboratories (Lexington, Ky.). Anti-Hsp90α antibodies for Western analysis (SPA-840) and for neutralizing function (SPS-771) were from Stressgen (Victoria, BC Canada). XL-10 Gold Ultra competent cells (XL-10 Gold) were from Stratagene (La Jolla, Calif.). pET system (pERT15b) for protein production in E. Coli was purchased from Novagen (Madison. WI). Brefeldin A (BFA) and dimethyl amiloride (DMA) were purchased from Sigma-Aldrich (St. Louis, Mo.). Matrigel invasion chambers (354480) and protocols were purchased from BD Biosciences (Bedford, Mass.). Athymic nu/nu mice (4-6 weeks of age, Harlan, Livermore, Calif.) were used in tumor formation assays.

### Hypoxia Treatment and Preparation of Serum Free Conditioned Media

The Oxycycler C42 from BioSpherix, Ltd (Redfield, N.Y.) was used as oxygen content controller throughout this study. This equipment allows creation of any oxygen profile with full range oxygen (0.1% to 99.9%) or CO₂ control (0.1% to 20.0%). More importantly, all media used for hypoxia experiments were pre-incubated in hypoxia chambers with the designated oxygen content for 16 hours, prior to the their use to replace normoxic culture media. Preparation of serum-free conditioned media was carried out as previously described (8).

### Lentiviral Systems for Up- or Down-Regulation of Target Genes

The pRRLsinhCMV system was used to overexpress exogenous HIF-1α. FG-12 RNAi delivery system was used to deliver shRNAs against HIF-1, as previously described (20). To measure protein levels of either endogenous or exogenous gene products, equal amounts (50 μg total cellular proteins/sample) of cell lysate proteins (measured by Bio-Rad Protein Assay) were subjected to antibody Western blot analyses. The results were visualized by ECL reactions. Films with unsaturated exposure were used for scanning densitometry. Means from three different exposures of the same experiment were calculated (42).

### Three Cell Migration Assay and Serum Free Conditioned Medium

Updated protocol for the colloidal gold cell motility assay, including data and statistic analysis, and a modified protocol for in vitro wound-healing assay, including pre-coating with an ECM, plating cells, scratching and quantifying migration data, were as described previously by us (29). Transwell assay was performed according our previously published protocol (21). Preparation of serum-free conditioned medium was as described in details previously (29). Zymography gel analysis was carried out as previously described (43).

### Invasion Assay

As described in details by the manufacturer's instruction for BD BioCoat™ Matrigel Invasion Chamber (354480) (BD Biosciences, Bedford, Mass.).

Densitometry with Alpha Innotech Fluorchem SP

The Alpha Innotech Fluorchem SP is a 4 Megapixel CCD specializing in fluorescence, chemiluminescence, or visible imaging. A 12 bit and 4 million pixel cooled camera is attached to a manual fixed lens in a MultiImage FC Light Cabinet (DE-500FC) with interference filter and ML-26 dual wavelength UV transilluminator. It uses Fluor Chem AlphaEase FC 32-bit software for image acquisition, enhancement, archiving, documentation, and analysis. A 1 min exposure with an aperture set at 1.2, zoom at 20 and focus at 1.9 with an open filter and normal sensitivity and high resolution, were used.

### Recombinant Hsp90α Production and Purification

The coding regions of seven of the eight distinct domains (Five, designated as N′, M-1, M-2, C′-1 and C′-2, were as previously reported, ref. 8) were subcloned into the His-tag pET15b vector (EMD biosciences, Inc., San Diego, Calif.) at BamHI using a PCR-based cloning technique. The 8th 27-amino acid peptide, P-9, was a synthetic peptide. The pET15b-hsp90α constructs were transformed into BL21-codonPlus (DE3)-RP competent cells (Stratagene) following the manufacturer-provided protocol. Protein synthesis was induced by the addition of 0.25 mM IPTG (Sigma, 15502-09) to the bacterium culture (O.D.≅0.8) and incubation for additional five hours at 25° C. These his-tagged proteins were first purified by Ni-NTA column with the HisBind purification kit (EMD biosciences, Inc.) according to the manufacturer's procedure. The purified proteins were concentrated in Amicon Ultra (10× or 50×) (Millipore, Billerica, Mass.) to ˜4 ml, filtered (0.22 μm) prior to load onto a Superdex-200 or 75 HiLoad gel filtration column (GE healthcare, Piscataway, N.J.) and separated by FPLC. The peptides were eluted by DPBS buffer (1.2 ml/min), concentrated in a Centricon YM-50 or YM-10 to 1 mg/ml and stored in 10% glycerol-DPBS at −70° C.

### Tumor Formation and Bioluminescent Imaging in Mice

Athymic nu/nu mice (4-6 weeks of age, Harlan, Livermore, Calif.) were implanted with three different cell lines (n=6/cell line) to determine the role of LRP-1/CD91 signaling in lung colonization of MDAMB-231 with control shRNA (against Lac-Z), MDAMB-231 LRP-1-RNAi and LRP-1^{-/-} MDAMB-468. Mice were anesthetized with 2% Isoflurane inhalant gas anesthesia using a vaporizer and injected with 1×10⁶ cells/mouse intravenously via the tail vein using custom made catheters to create the metastatic model. All animal experiments were performed in accordance to the protocol approved by the USC Institutional Animal Care and Use Committee (IACUC). Optical imaging was performed using the IVIS 200 Imaging System (Xenogen Corporation, Alameda, Calif.), which utilizes a cooled CCD camera for optimized sensitive, low light-level in vivo imaging. Mice were anesthetized throughout the study using 2% Isoflurane inhalant gas anesthesia followed by an intraperitoneal injection of the luciferase substrate, D-luciferin (50 mg/kg, Caliper Life Sciences, Alameda, Calif.).

Distribution of the substrate occurred for 12 minutes, followed by bioluminescent imaging with the following settings: 1 minute/scan, bin=8, field of view 13.1 cm, and f-stop=1. Mice were imaged sequentially in both dorsal and ventral views during the bioluminescent signal plateau phase and analyzed using the Living Image 3.1 Software (Xenogen Corporation, Alameda, Calif.). Mice were imaged weekly until IACUC endpoints were met. All images were normalized to the same image pseudo-color scale and circular regions of interest (ROI) were drawn over the chest area to quantify the bioluminescent signal. Data from the MDAMB-231-vector group were compared using a Wilcoxon rank-sum test for statistical significance (p<0.05) and qualitatively compared to the distribution of signal from the MDAMB-231-LRP-1-RNAi and LRP-1^{-/-} MDAMB-468 groups. The above experiment was repeated three times.

### Histochemistry

Whole lung with or without primary tumors were dissected, fixed in 10% formalin (Sigma), embedded in paraffin, cut into 6 μm sections throughout the entire lung and stained with hematoxylin and eosin (H&E). Slides were analyzed and the tumors counted through the whole lung section on slides using a microscope and quantified.

### Statistical Analyses

Cell migration and invasion data are presented as mean standard deviation (s.d.). All the in vitro cell experiments were analyzed with a two-tailed Student's t-test with a CI (confidence interval) of >90%. Analysis of the lung colonization experiment data (photons/sec.) was performed using the two-tailed nonparametric Mann-Whitney test. p values less than 0.05 were considered statistically significant.

### Example 2

### A Constitutive Level of HIF-1α is Essential for Invasiveness of Breast Cancer Cells

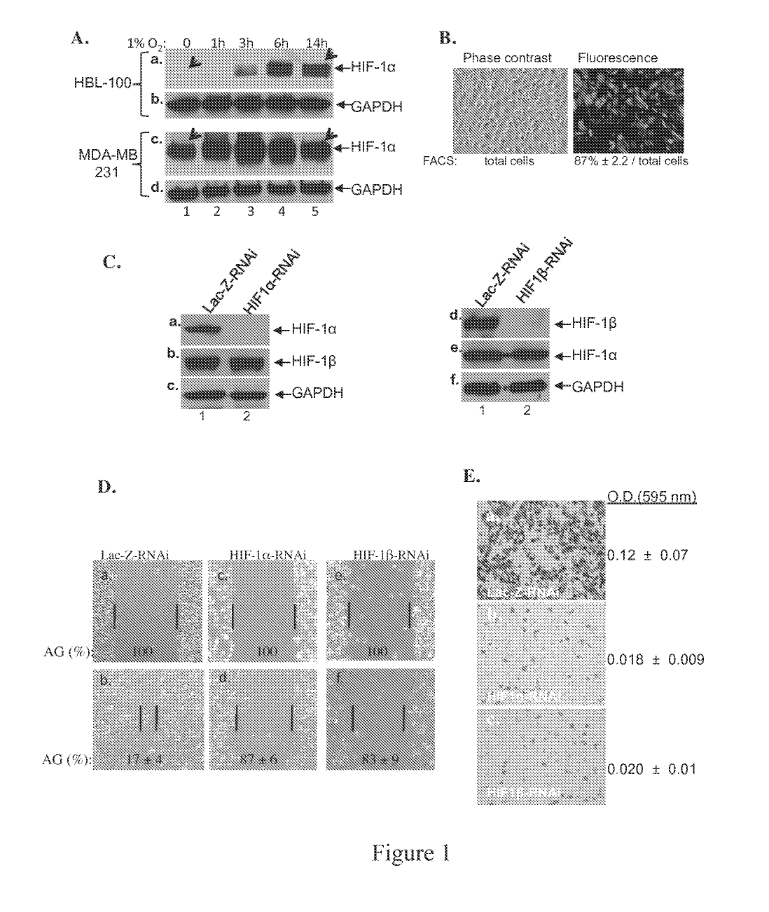

Applicants wanted to identify a tumor cell line with deregulated expression of HIF-1α and use this cell model for identifying new downstream effectors of the deregulated HIF-1α essential for cell invasion in vitro and tumor formation in vivo. After screening various tumor cell lines (listed in Materials and Methods), Applicants focused on the ER-negative and Ah-nonresponsive breast cancer cell line, MDA-MB-231, previously isolated from pleural effusion obtained from 51-years old patient, invasive and metastatic (4). This choice also considered the clinical data that approximately 30% of invasive breast cancer samples are hypoxic (10, 22). The untransformed human epithelial cells, HBL-100 (13), were included as a control. As shown in FIG. 1A, in HBL-100 cells, the HIF-1α level was undetectable under normoxia (panel a, lane 1). A time-dependent accumulation of HIF-1α protein was detected from the cells under hypoxia (lanes 2-5). Under identical conditions (equal protein loading, side-by-side operations and ECL processes), however, a constitutive basal level of HIF-1α expression could be detected in MDA-MB-231 cells even under normoxia (panel c, lanes 1). While hypoxia treatment of the cancer cells caused further increase in HIF-1α, such increase was transient. It reached a plateau between 3-6 hours and then declined to the basal level by 14 hours under hypoxia (lanes 2-5 and data not shown). While the significance of this short-term induction of HIF-1α in response to hypoxia in MDA-MB-231 cells remains unknown, the constitutive presence of HIF-1α is consistent with the increased anti-HIF-1 antibody staining of human breast tumor tissue specimens (10, 22).

The constitutive level of HIF-1α in MDA-MB-231 cells is sufficient for maintaining a high motility and invasiveness of the cells even under serum-free conditions (a mimic of the hypoxic tumor environment in vivo). Applicants used the lentiviral system, FG-12, to deliver an U6 promoter-driven shRNA against human HIF-1α or HIF-1β into MDA-MB-231 cells. Expression of a CMV promoter-driven GFP (green fluorescent protein) gene in the same vector, as shown in FIG. 1B, indicated a more than 80% gene transduction efficiency in this cell line by this system (right panel versus left panel). Therefore, as shown in FIG. 1C, Applicants achieved nearly complete down-regulation of HIF-1α (panel a, lane 2) or HIF-1β (panel d, lane 2), in comparison to control shRNA against LacZ (lanes 1). Moreover, neither shRNA cross-reacted non-specifically between HIF-1α and HIF-1β (panels b and e). When these HIF-1α- or HIF-1β-down-regulated cells were subjected to cell motility (“scratch”) assays, as shown FIG. 1D, the control MDA-MB-231 cells exhibited a constitutively high motility even under serum-free conditions (panel b vs. panel a). However, downregulation of HIF-1α or HIF-1β paralyzed the cell motility (panels d and f vs. panels c and e). Similar observations were made in invasion assays of the same cells. As shown in FIG. 1E, control MDA-MB-231 cells strongly penetrated a matrigel barrier (consisting of laminin, type IV collagen, heparan sulfate proteoglycan and nidogen) under serum-free conditions (panel a). In contrast, the HIF-1α- or HIF-1β-downregulated cells exhibited dramatic reduction in invasion (panels b and c vs. panel a). These results establish MDA-MB-231 cell line as an adequate tumor cell model to identify downstream effectors of deregulated HIF-1α.

### Example 3

### Deregulated HIF-1α in Breast Cancer Causes Constitutive Hsp90α Secretion

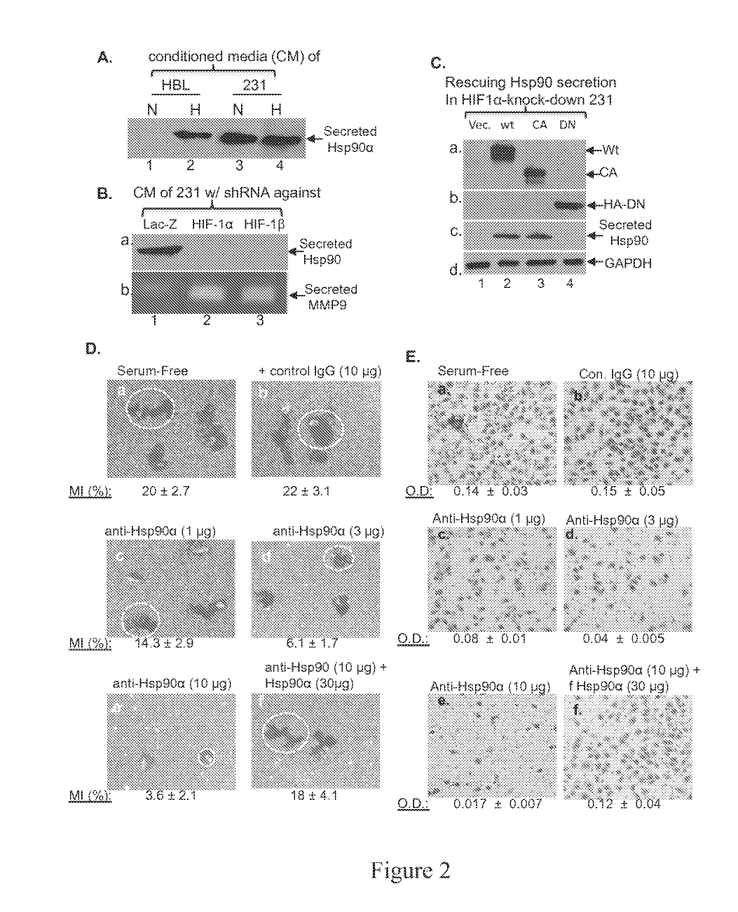

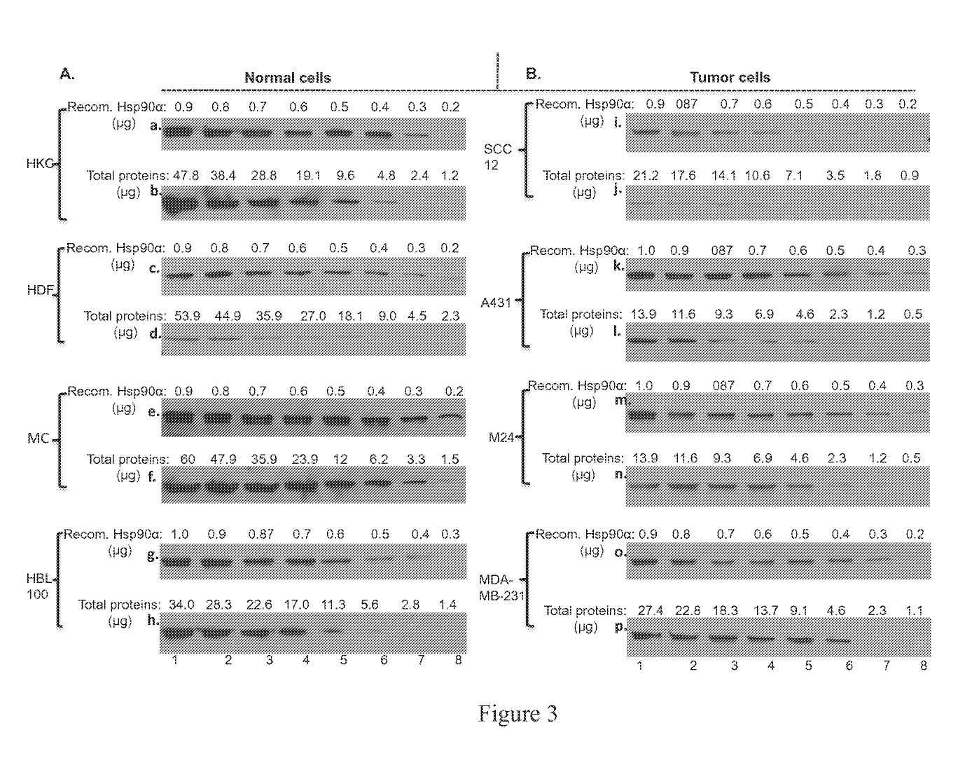

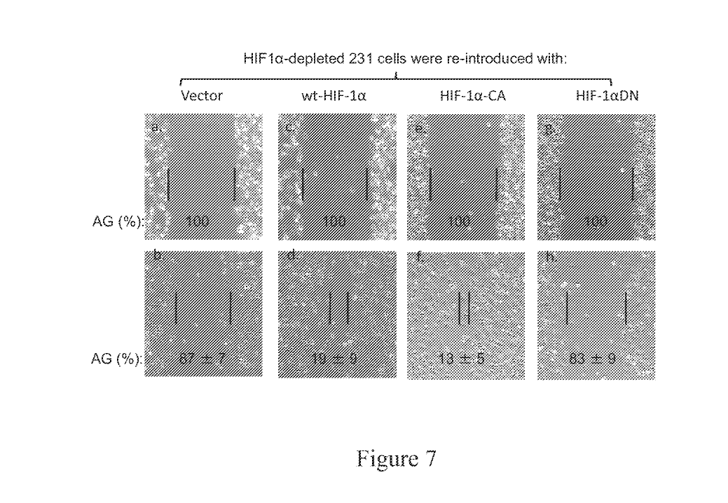

Applicants conducted a search for targets of deregulated HIF-1α for three criteria: 1) constitutively secreted by MDA-MB-231 cells, 2) under direct control of the deregulated HIF-1α and 3) essential for the invasiveness of the cells. Applicants focused on secretion of heat shock protein-90α (Hsp90α) based on following evidence. First, Eustace et al showed that HT-1080 fibrosarcoma cells secrete Hsp90α (12). Second, hypoxia causes normal cells to secrete Hsp90α (19). Third, secreted Hsp90α mediates hypoxia-driven cell migration (42). Therefore, Applicants tested the possibility that the deregulated HIF-1α causes Hsp90α secretion, leading to increased migration and invasion of MDA-MB-231 cells. Serum-free conditioned media (CM) of HBL-100 and MDA-MB-231 cells cultured under either normoxia or hypoxia were analyzed for the presence of Hsp90α. As shown in FIG. 2A, secreted Hsp90α was detected from the CM of HLB-100 cells incubated under hypoxia (lane 2), but not normoxia (lane 1). In contrast, equal amounts of secreted Hsp90α protein were detected from CM of MDA-MB-231 cells cultured under either normoxia (lane 3) or hypoxia (lane 4). Applicants found that the constitutive secretion of Hsp90α was caused by the deregulated HIF-1α in the cells. It is shown in FIG. 2B that, while constitutive Hsp90α secretion remained unaffected in Lac-Z-RNAi infected MDA-MB-231 cells (a, lanes 1), secreted Hsp90α was undetectable from the CM of either HIF-1α- or HIF-1β-down-regulated MDA-MB-231 cells (a, lanes 2 and 3). This inhibition appeared to be specific, since under identical conditions, secretion of MMP9 is rather increased (b, lanes 2 and 3 vs. lane 1). Applicants point out that, unlike intracellular proteins (like β-actin or GAPDH for intracellular proteins), there are limited reliable loading control markers for secreted proteins. The equal loadings of CM were justified by taking equal volumes of CM from the same number of cells cultured under identical conditions. To further validate the specificity of HIF-1α action, Applicants carried out HIF-1α gene rescue experiments in endogenous HIF-1a-depleted MDA-MB-231 cells. As shown in FIG. 2C, Applicants exogenously re-introduced wild type (wt) and constitutively active (CA) of HIF-1α into these cells, as detected by anti-HIF-1α antibody immunoblotting analysis (panel a, lanes 2 and 3 vs. lane 1). Since DN-HIF-1α has a large deletion in the region most commercial anti-HIF-1α antibodies recognize (20), Applicants instead proved the expression of the 34-kDa DN-HIF-1α by using anti-HA tag antibody blotting (panel b, lane 4). From CM of these cells, Applicants found that only wt-HIF-1α and CA-HIF-1α were able to rescue Hsp90α secretion (panel c, lanes 2 and 3 vs. lane 1), but not for DN-HIF-1α (lane 4). These results indicated that secreted Hsp90α is a direct downstream target for deregulated HIF-1α. More importantly, the wt-HIF-1α and CA-HIF-1α, but not DN-HIF-1α, were also able to rescue the defect of cell motility of the endogenous HIF-1α-depleted cells (FIG. 7).

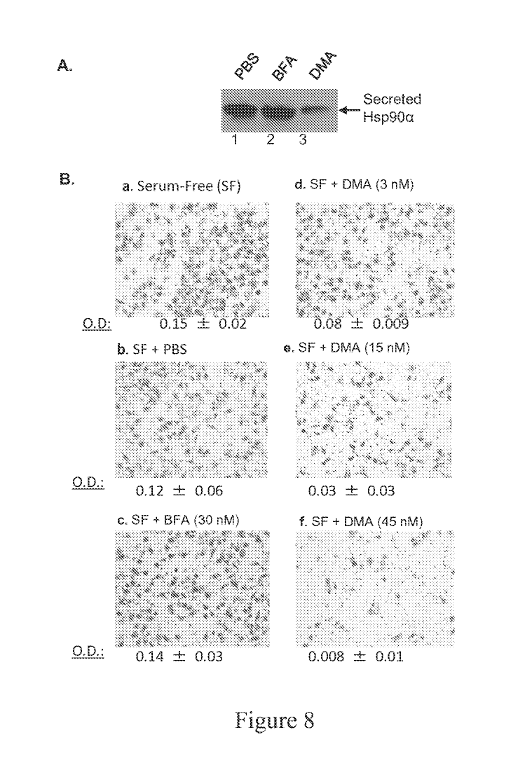

Next, the Applicants examined whether secreted Hsp90α mediates the deregulated HIF-1α-driven MDA-MB-231 cell migration and invasion. Applicants took approach of using a neutralizing antibody against secreted (not intracellular) Hsp90α. To test cell motility, Applicants switched to use the colloidal gold migration assay, because this assay measures motility of individual (instead of a population of) cells and is particularly relevant to study of an autocrine signaling mechanism. As shown in FIG. 2D, MDA-MB-231 cells already exhibited a strong motility even under serum-free conditions (panel a). The addition of a control IgG showed little effect (panel b vs. panel a). However, the addition of an increasing amount of the neutralizing antibody against Hsp90α into the medium reduced the constitutive motility of MDA-MB-231 cells in a dose-dependent manner (panels c to e). The addition of excess amounts of recombinant Hsp90α protein reversed inhibition of the cell migration by the neutralizing antibody (panel f). More convincingly, the same neutralizing antibody also blocked the ability of MDA-MB-231 cells to invade through a Matrigel barrier. As shown in FIG. 2E, the addition of an increasing amount of the antibody blocked MDA-MB-231 cell invasion in a dose-dependent fashion (panels c to e), in comparison to medium alone (panel a) or medium with control IgG (panel b). Again, the addition of excess amount of recombinant Hsp90α protein reversed the inhibition of MDA-MB-231 cell invasion by anti-Hsp90α antibodies (panel f). These findings were further confirmed by a pharmacological approach. As shown in FIG. 8, treatment of the cells with dimethyl amiloride (DMA), a specific inhibitor of protein secretion via the exosome protein trafficking pathway (8), blocked Hsp90α secretion (A, lane 3 vs. lane 1) and MDA-MB-231 cell invasion in a dose-dependent manner (B, panels d, e, f). In contrast, Brefeldin A (BFA), an inhibitor of the classical ER-Golgi protein trafficking pathway (8), showed little effect on Hsp90α secretion (A, lane 2 vs. lane 1) or cell invasion (B, panels c). Taken together, it was concluded that the deregulated HIF-1α controls Hsp90α secretion and uses the secreted Hsp90α to promote breast cancer cell migration and invasion in the absence of any exogenous growth factors.

### Example 4

### Comparisons of Hsp90α Stored in Tumor Cell Versus Normal Cells

The three original reports, which are cited for the widely used statement of “1-2% of total cellular proteins”, included an abstract and two research articles (5, 14, 40). Applicants were unable to confirm the origin of the statement. None of these publications contained any direct supporting data for that statement. In fact, none of them even made that statement. Since it is critical to understand how much Hsp90α a tumor cell has, Applicants took a multi-step approach to re-establish the amount (%) of Hsp90α protein in reference to the total cellular proteins in four normal and four cancer cells. 1) Applicants used a series of known amounts of bovine serum albumin (BSA, ˜55 kDa) to establish a standard curve of O.D. readings versus the actual amounts (μg) of the protein. 2) An increasing volume of total post-nuclear extract of a given cell type was subjected to the O.D. reading and, then, converted the reading into μg of proteins according to the Standard Curve. 3) These cell extracts with known μg of proteins and a series of known μg of recombinant human Hsp90α proteins were subjected side-by-side to SDS-PAGE and Western blot with a monoclonal anti-Hsp90α antibody. Note: the entire processes of SDS-PAGE electrophoresis, transfer onto nitrocellulose membranes, blotting, primary antibody, secondary antibody, washing and ECL reactions of the two sets of samples for a given cell type were operated all in common apparatus, containers and the same exposure cassette for the exactly same period of time. 4) The Hsp90α protein bands from the cell lysates and the recombinant Hsp90α, as shown in FIG. 3, were subjected to densitometry scanning with identical settings of parameters (Alpha Innotech Fluorchem SP). 5) Applicants then used the scanning readings of the recombinant Hsp90α bands to establish a second Standard Curve that measures densitometry readings versus the actual amounts (μg) of the recombinant Hsp90α protein. 6) Using this standard curve, the scanning readings of the Hsp90α bands from the total cell extracts were converted to amount (μg) of proteins. 7) Finally, the amount of Hsp90α (μg) in a given volume of cell extract is divided by total proteins (μg) from the same extract ×100=% of Hsp90α in the total proteins of the given cell type. The means of calculated numbers from up to eight sets of Hsp90α: cell extract pairs was taken as the final estimated % of Hsp90α for a given cell type (Table 1). Applicants found that 1) Hsp90α protein accounts for 2-3% of the total cellular proteins in the normal cells tested and it goes up to 7% in certain tumor cell lines and 2) the intracellular level of Hsp90α is not elevated in all cancer cells versus their normal counterpart, such as the difference in Hsp90α between MDA-MB-231 and HBL-100 cells.

TABLE 1
Estimated percentage of Hsp90α In normal versus tumor cells*

| Normal cells | | | Cancer cells | | |
|---|---|---|---|---|---|
| Name | Origin | % of Hsp90α | Name | Origin | % of Hsp90α |
| HKC | human | 2.35% ± 0.26 | SCC-12 | Scranous | 3.29% ± 0.81 |
| | keratinocyte | | | carcinoma | |
| MC | human | 2.25% ± 0.62 | M-24 | human | 6.76% ± 1.06 |
| | melanocyte | | | melanoma | |
| HDF | human skin | 2.33% ± 0.16 | A431 | Dermal | 4.53% ± 0.70 |
| | fibroblast | | | carcinoma | |
| HBL-100 | human breast | 3.47% ± 0.24 | MDA-MB-231 | human breast | 3.66% ± 0.21 |
| | epithelial | | | cancer | |

*The data (%) for each cell line was the result of calculation of three Independent experiments (i.e. from cell culture to densitometry). The total protein amount In cell lysate (with proteins of varieties of molecular mass) was an estimate, due to the standard curve made of a single 55-kDa protein (BSA). Based on statistic significance below p < 0.05, the difference between HK versus SCC and between HBL-100 and MDA-MB-231 are not significant.

### Example 5

### Identification of the Therapeutic Epitope in Secreted Hsp90α Critical for Mediating the Deregulated HIF-1α-Driven Invasion

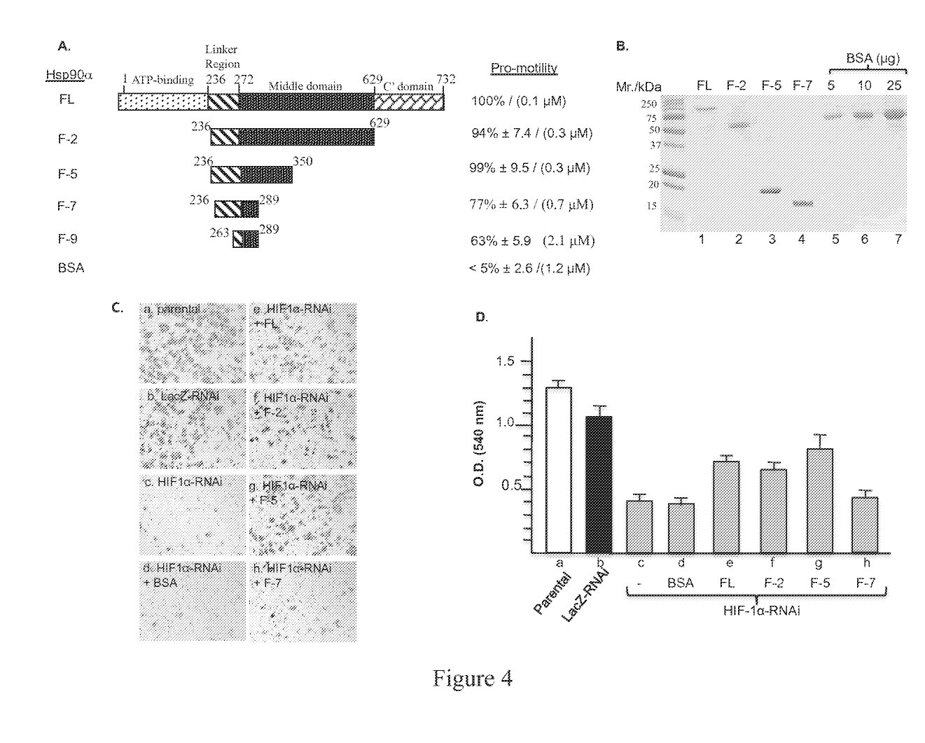

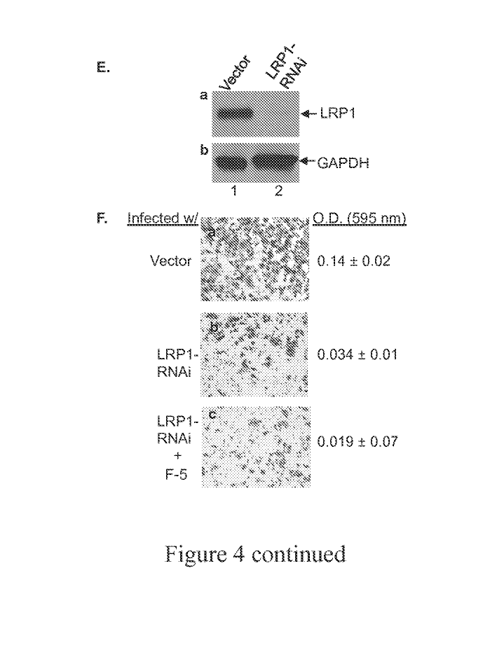

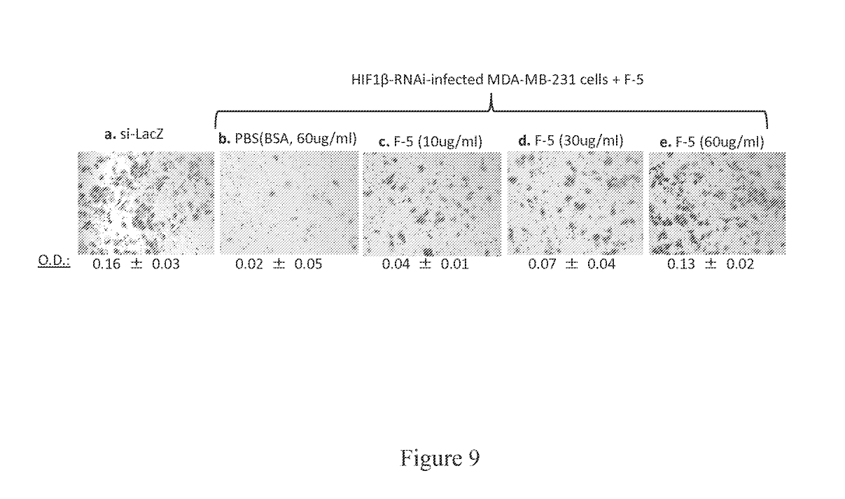

To identify the therapeutic target in secreted Hsp90α, Applicants used systematic mutagenesis to narrow down the minimum functional epitope in human Hsp90α. Initially, eight recombinant peptides of Hsp90α were generated and tested for stimulation of cell motility under serum-free conditions. Five of the eight peptides (FL, F-2, F-5, F-7 and F-9), which retained various degrees of the full pro-motility by the full-length Hsp90α, are schematically shown in FIG. 4A and the purity of these peptides was analyzed by SDS-PAGE, as shown in FIG. 4B. F-9 is a 27-amino acid synthetic peptide (too small to show on SDS gel). Since down-regulation of the endogenous HIF-1α blocked Hsp90α secretion and Matrigel invasion by MDA-MB-231 cells, Applicants reasoned that supplementation of a functional peptide should bypass the blockade of HIF-1 down-regulation and rescue the invasion defect of the cells. This rescue approach should allow us to identify the minimum epitope in secreted Hsp90α. As shown in FIG. 4C, the parental and control LacZ-RNAi-infected MDA-MB-231 cells showed strong invasion (panels a and b), whereas HIF-1α-downregulated MDA-MB-231 cells were unable to invade (panels c). Supplementation of BSA could not rescue the invasion defect (panel d). However, FL, F-2 and F-5 under their pre-determined optimized concentrations were able to partially rescue the invasion defect of the cells (panels e-g). In contrast, F-7 (panel h) and F-9 (data not shown) showed little rescuing effects, even if they still retained significant pro-motility activity. Quantitation of the data, as shown in FIG. 4D, revealed that FL, F-2 and F-5 were equally effective (bars e, f, g). F-7 was virtually unable to rescue (bar h). Similar results were obtained in HIF-1β-downregulated MDA-MB-231 cells, in which F-5 rescued the invasion defect of the cells (FIG. 9). To further confirm that the rescuing mechanism by these peptides was to bypass the blockade of HIF-1α downregulation, Applicants found that supplementation of F-5 was unable to rescue the invasion defect of MDA-MB-231 cells with down-regulated LRP-1, the receptor for extracellular Hsp90α signaling (8, 42). As shown in FIG. 4E, Applicants achieved a near complete downregulation of LRP-1 by the FG-12 system (lane 2 vs. lane 1), which resulted in dramatic reduction of the cell invasion (FIG. 4F, panel b vs. panel a). The addition of F-5 was unable to rescue the invasion defect due to downregulation of LRP-1 (panel c).

### Example 6

### Interruption of Hsp90α Autocrine Signaling Blocks Breast Cancer Cell Lung Colonization and Tumor Formation

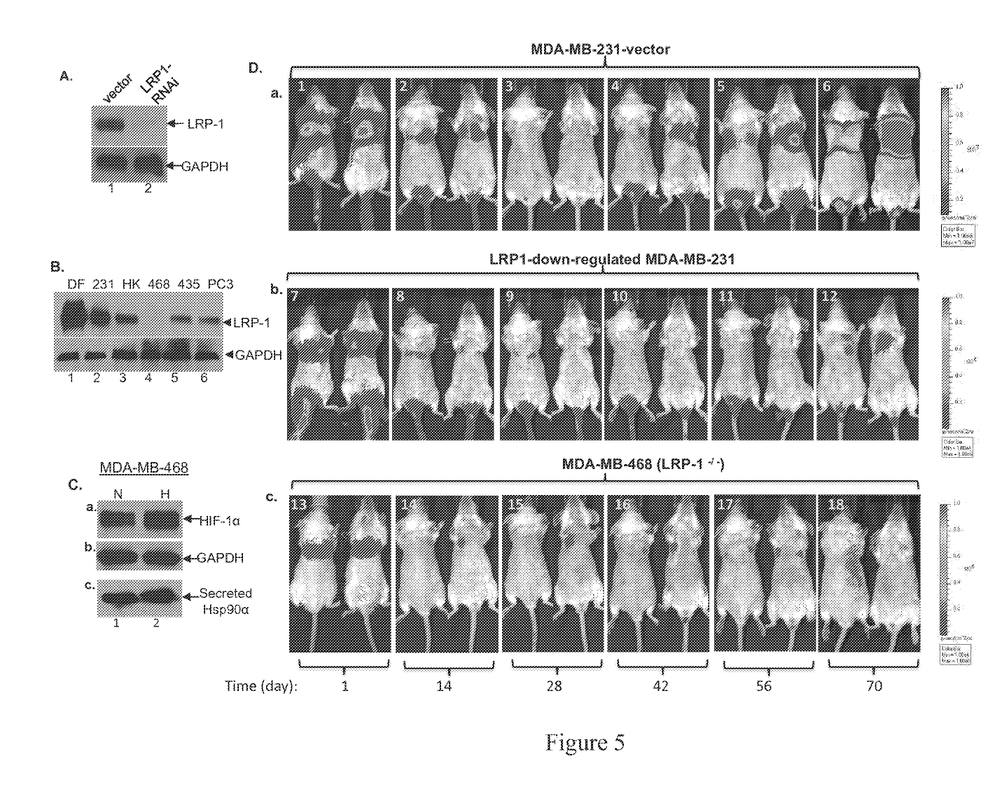

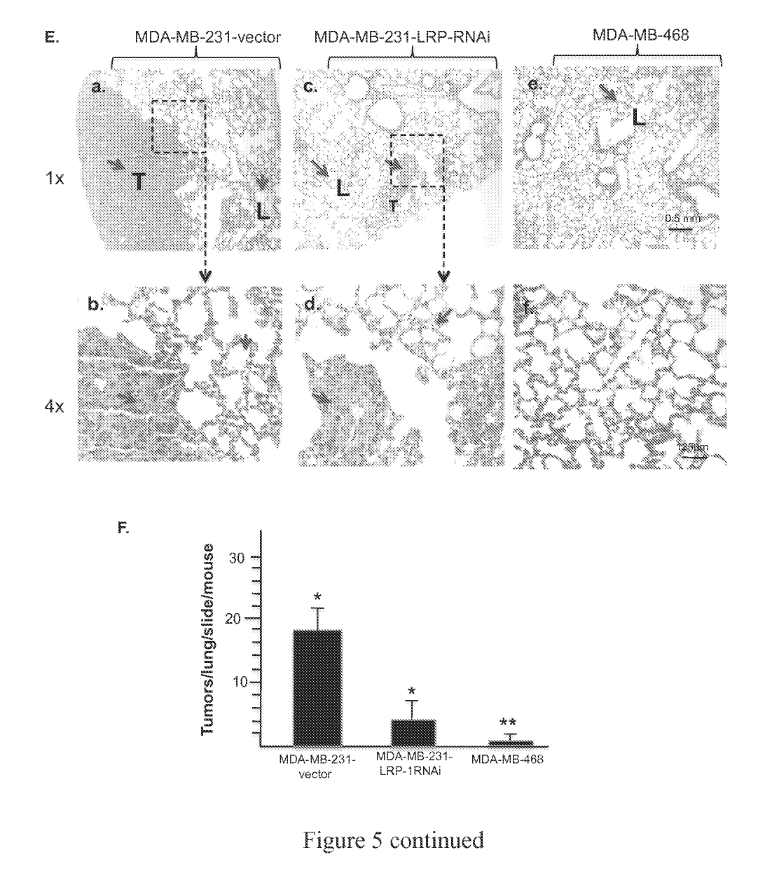

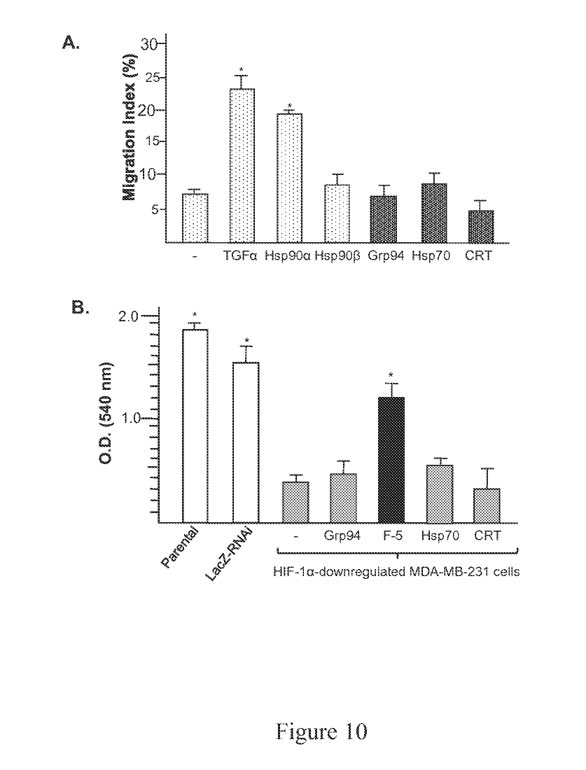

Colonization of various secondary organs by breast cancer depends upon productive interactions between the tumor cells and the stromal microenvironment. Lung colonization assay in nude mice is an accepted model to test these abilities of a tumor cell. Currently, there is lack of effective and specific inhibitors against secreted Hsp90α actions in vivo. Genetic knockdown of the entire Hsp90α would not distinguish between intracellular and extracellular Hsp90α. Injection of membrane-impermeable Geldanamycine (GM)-based inhibitors was unstable and toxic to the animal and was hard to distinguish direct versus indirect effects of the drug whence they enter the circulation (39). Having considered these limitations, Applicants took the approach of targeting an immediate downstream step of the secreted Hsp90α signaling, the LRP-1 receptor. FG-12 system was used to make a permanent knockdown of LRP-1 in MDA-MB-231 cells. As shown in FIG. 5A, nearly complete downregulation of LRP-1 was verified in the exact MDA-MB-231 cells 24 hours prior to injection (a, lane 2 vs. lane 1). In addition, Applicants found that, while MDA-MB-231 and several other cell lines all showed LRP-1 positive, the MDA-MB-468 breast cancer cells lost LRP-1 expression (FIG. 5B, lane 4). However, like MDA-MB-231 cells, MDA-MB-468 cells also maintain a constitutive HIF-1α expression and constitutive Hsp90α secretion (FIG. 5C, panels a and c). Thus, the inventors included MDA-MB-468 cells as naturally occurring LRP-1-null cells. Both of these cell lines were pre-engineered to stably express a luciferase gene and injected via tail vein into circulation of SCID mice. As shown in FIG. 5D, after an initial lung homing observed for all three cell lines (panels 1, 7 and 13), the majority of the cells became either weakly detectable or undetectable for the subsequent 4 weeks (panels 2 and 3; 8 and 9, 14 and 15). Then, the vector-infected MDA-MB-231 cells returned to develop tumors in lung around 42 days after injection in 6 out of 7 mice/group (panels 4-6). In contrast, LRP-1-knockdown MDA-MB-231 cells showed much reduced ability of lung colonization (panels 10, 11, 12), which occurred only in 2 out of 7 mice/group. MDA-MB-468 cells showed little lung metastasis in all mice. When dissecting the mice on day 70, Applicants found visible tumors only in lungs of the mice injected with vector-MDA-MB-231, but not with LRP-1-knockdown MDA-MB-231 cells or MDA-MB-468 cells. Hematoxylin and eosin (H&E) staining of sections across the entire lung tissue, as shown in FIG. 5E, showed large invading tumors in the lungs of the mice injected with vector-MDA-MB-231 (a and b), much smaller and less number tumors in lungs of the mice injected with LRP-1-knockdown MDA-MB-231 cell (c and d) and no visible tumors in the lungs of the mice injected with MDA-MB-468 cell (e and f). Quantitation of these data is shown in FIG. 5F. LRP-1 (also called CD91) is a large heterotrimeric protein consisting of a 515-kDa extracellular domain and an 85-kDa trans-membrane subunit (17). Besides Hsp90α, several other extracellular heat shock proteins were reported to also bind LRP-1, including secreted calreticulin (CRT), gp96 and Hsp70 (2). Therefore, the effect of LRP-1-knockdown on tumor formation did not necessarily prove that the reduced tumor formation was specifically due to blockade of secreted Hsp90α signaling through LRP-1. To address this issue, Applicants tested whether recombinant Hsp70, gp96 and CRT promote cell migration and if they could rescue, like Hsp90α, the invasion defect of HIF-1α-down-regulated MDA-MB-231. In both migration and invasion assays, Hsp70, gp96 and CRT showed limited effects (FIG. 10). Taken together, these findings support the hypothesis that the “HIF-1>secreted Hsp90α>LRP-1” autocrine loop plays a critical role in deregulated HIF-1α-driven tumor rogression.

### Example 7

### Discussion

Breast cancer affects approximately one out of every nine women in the US, making it the most common female malignancy (30). Breast cancer was traditionally divided into a few well-established tumor types according to their microscopic appearance and growth behaviors. For instance, the hormone status of breast cancers includes estrogen receptor (ER) positive (relies on estrogen for growth), progesterone receptor (PR) positive (relies on progesterone for growth) and hormone receptor (HR) negative (doesn't have hormone receptors, so it doesn't need hormones for growth). The death rate from the disease has dropped modestly over the past decade due to availability of multiple treatment choices:

surgery, radiation, hormonal therapy and chemotherapy. However, there has been a growing sense of frustration among breast cancer experts over side effects and unsustainable results of current treatments, such as resistance of hormone receptor positive breast cancers to endocrine therapies. It is clear now that every breast cancer is genetically unique. Many of the genetic differences between individual tumors influence the likelihood that the cancer will recur. These differences are associated in part with expression of various groups of cancer-related genes (27). Therefore, there is a pressing need to identify more common markers for diagnosis and treatments.

Dales et al carried out anti-HIF-1α immunohistochemical assays on frozen sections of 745 breast cancer samples and found that the levels of HIF-1α expression correlated to poor prognosis, lower overall survival and high metastasis risk among both node-negative and node-positive patients (10). By using HIF-1α expression as a marker, it was estimated that approximately 25-40% of all invasive breast cancer samples are hypoxic, suggesting that HIF-1α can be used as a broader marker for breast cancers. In the current study, Applicants have identified a critical downstream effector of HIF-1α in breast cancer cells, the secreted Hsp90α. Furthermore, Applicants have identified a 115-amino acid peptide, F-5, in Hsp90α that is necessary and sufficient for the HIF-1α-mediated breast cancer cell invasion.

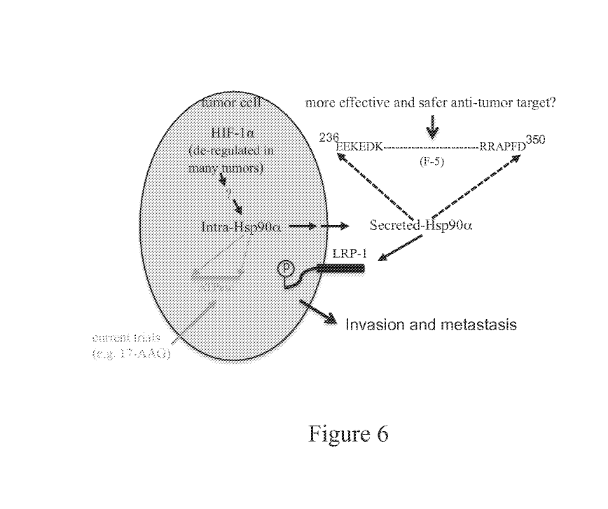

Applicants propose that the F-5 region of Hsp90α may prove to be more effective and less toxic for treatment of breast cancer patients. This hypothesis is based on studies of past seven years. Li et al showed that normal skin fibroblasts do not secrete Hsp90α under normoxia and secrete Hsp90α under hypoxia (19). Eustace et al showed that tumor cells constitutively secrete Hsp90α (12). Based on these findings, Applicants further propose a shift of anti-cancer strategy from targeting the intracellular Hsp90α, such as by 17-AAG, to targeting the secreted Hsp90α at the F-5 region. The overall hypothesis is schematically summarized in FIG. 6.

For many years, toxicity versus efficacy has been a long-standing challenge for clinical trials of the anti-cancer drugs targeting the ATPase of intracellular Hsp90, i.e. 17-AAG and its derivatives. These inhibitors need to selectively harm the overly active or overexpressed Hsp90α in cancer cells that embed in normal tissues, while to minimize any damage of the physiological chaperone functions of Hsp90 in the surrounding normal cells. In contrast, no physiological processes (gene expression, metabolism, proliferation and development) have been reported to require secretion of Hsp90α by the cells. Instead, secretion of Hsp90α appears to be an “emergency response” of normal cells to environmental insults, such as tissue injury, hypoxia, and irradiation. However, genetic alterations in cancer cells use constitutively expressed HIF-1α to trigger Hsp90α secretion and use the secreted Hsp90α to cope with otherwise non-livable environment for normal cells (7). Therefore, Applicnats propose that new anti-cancer drugs that selectively target the F-5 epitope should achieve higher efficacy and pose less toxicity to the cancer patients.

What is the downstream target of secreted Hsp90α? Eustace and colleagues reported that Hsp90α, but not Hsp90β, promotes cancer cell migration and invasion by binding to and activating the matrix metalloproteinase 2 (MMP2) (12). Sidera and colleagues showed that a pool of cell membrane-bound Hsp90α interacts with HER-2 tyrosine kinase receptor in breast cancer cells, leading to increased MMP2 activation, cell motility and invasion (31). Recently, Gopal and colleagues reported that extracellular Hsp90α stimulates a binding of LRP-1 to EphA2 receptor during glioblastoma cell invasion (15). Cheng et al used four independent approaches (neutralizing antibodies, RAP inhibitor, RNAi, and somatic LRP-1-negative mutant cell line), to demonstrate that the widely expressed cell surface receptor, LRP-1, mediates the extracellular Hsp90α signaling (8). In the current study, Applicants show that Hsp90α failed to stimulate migration and invasion of LRP-1-downregulated MDA-MB-231 cells in vitro, consistent with the report by Song et al that LRP-1 is required for glioblastoma cell migration and invasion in vitro (34). Furthermore, LRP-1-down-regulated MDA-MB-231 or LRP-1-null MDA-MB-468 cells exhibited dramatic reduced lung colonization and tumor formation in vivo. Understanding how each of the target proteins contributes to Hsp90α-stimulated invasion would require simultaneous studies of these molecules in a common cell system.

Despite the various experimental approaches, demonstration of the role of secreted Hsp90α in tumor progression in vivo may still require availability of more stable and specific inhibitors. Tsutsumi and colleagues utilized DMAG-N-oxide, a geldanamycin/17-AAG-derived and cell membrane-impermeable Hsp90 inhibitor, to pre-treat melanoma cells to block extracellularly located Hsp90α, prior to injecting them into nude mice. They reported that DMAG-N-oxide treated cells showed decreased motility and invasion of the cells in vitro and reduced lung colonization in vivo (39). There are several limitations of this approach. First, 17-AAG binds and inhibits the ATPase activity of Hsp90. However, Cheng et al has demonstrated that the ATPase domain is dispensable for the extracellular action of Hsp90 (8). Therefore, the inhibitory effect of 17-AAG is not a direct inhibition of the functional epitope in extracellular Hsp90's. Second, it is hard to understand how a single pre-treatment of the cells with the drug in vitro (due to the drug's structurally unstable in vivo) could have had the reported long lasting effect after the cells were injected into mice. Patsavoudi and colleagues reported development of a monoclonal antibody, 4C5, that neutralizes secreted Hsp90α and Hsp90β in vitro. They reported that melamona or breast cancer cells mixed with 4C5 in vitro showed reduced lung colonization, in comparison to mixing with a control antibody, after the cells were injected into nude mice (34, 38). However, it is hard to imagine that the co-injected 4C5 could have worked by continuously binding and neutralizing the constantly secreted Hsp90α and Hsp90β by the tumor cells for the entire period of the multi-week experiment. It would make more sense to inject and maintain a steady-state amount of 4C5 in circulation, prior to injection with tumor cells. Applicants showed that breast cancer cells lacking the LRP-1 receptor were unable to effectively form tumors in nude mice. As pointed out earlier, the effect of down-regulation of LRP-1 may not necessarily be due to a specific blockade of secreted Hsp90α signaling, since LRP-1 may potentially bind other unidentified ligands. Therefore, identification of the F-5 peptide in secreted Hsp90α provides an excellent target for design of new, effective and more specific inhibitors for studying the role of tumor-secreted Hsp90α and even their therapeutic potentials.

### REFERENCES

1. Arany, Z., Huang, L. E., Eckner, R., Bhattacharya, S., Jiang, C., Goldberg, M. A., Bunn, H.F., and Livingston, D. M. (1996). An essential role for p300/CBP in the cellular response to hypoxia. Proc Natl Acad Sci USA 93, 12969-12973.
2. Basu, S., Binder, R. J., Ramalingam, T., and Srivastava, P. K. (2001). CD91 is a common receptor for heat shock proteins gp96, hsp90, hsp70, and calreticulin. Immunity 14, 303-313.
3. Bertout, J. A., Patel, S. A., and Simon, M. C. (2008). The impact of O₂ availability on human cancer. Nat Rev Cancer 8, 967-975.
4. Cailleau, R., Young, R., Olive, M., and Reeves, W. J., Jr. (1974). Breast tumor cell lines from pleural effusions. J Natl Cancer Inst 53, 661-674.
5. Catelli, M.-G., Joab, I., Renoir, J.-M., Mester, J., Binart, N., Radanyi, C., Buchou, T., Zoorob, R., and Baulieu, E.-E. (1983). 402 Immunological studies of chick oviduct progesterone receptor. J. Steroid Biochem. 19, 134.
6. Chen, B., Piel, W. H., Gui, L., Bruford, E., and Monteiro, A. (2005). The HSP90 family of genes in the human genome: insights into their divergence and evolution. Genomics 86, 627-637.
7. Cheng, C. F., Fan, J., Fedesco, M., Guan, S., Li, Y., Bandyopadhyay, B., Bright, A. M., Yerushalmi, D., Liang, M., Chen, M., Han, Y. P., Woodley, D. T., and Li, W. (2008). Transforming growth factor alpha (TGFalpha)-stimulated secretion of HSP90alpha: using the receptor LRP-1/CD91 to promote human skin cell migration against a TGFbeta-rich environment during wound healing. Mol Cell Biol 28, 3344-3358.
8. Cheng, C. F., Fan, J., Zhao, Z., Woodley, D. T., Li, W. (2010). Secreted Heat Shock Protein-90alpha: A More Effective and Safer Target for Anti-Cancer Drugs? Current Signal Transduction Therapy 5, 121-127.
9. Csermely, P., Schnaider, T., Soti, C., Prohaszka, Z., and Nardai, G. (1998). The 90-kDa molecular chaperone family: structure, function, and clinical applications. A comprehensive review. Pharmacol Ther 79, 129-168.
10. Dales, J. P., Garcia, S., Meunier-Carpentier, S., Andrac-Meyer, L., Haddad, O., Lavaut, M. N., Allasia, C., Bonnier, P., and Charpin, C. (2005). Overexpression of hypoxia-inducible factor HIF-1alpha predicts early relapse in breast cancer: retrospective study in a series of 745 patients. Int J Cancer 116, 734-739.
11. Denzer, K., Kleijmeer, M. J., Heijnen, H. F., Stoorvogel, W., and Geuze, H. J. (2000). Exosome: from internal vesicle of the multivesicular body to intercellular signaling device. J Cell Sci 113 Pt 19, 3365-3374.
12. Eustace, B. K., Sakurai, T., Stewart, J. K., Yimlamai, D., Unger, C., Zehetmeier, C., Lain, B., Torella, C., Henning, S. W., Beste, G., Scroggins, B. T., Neckers, L., Ilag, L. L., and Jay, D. G. (2004). Functional proteomic screens reveal an essential extracellular role for hsp90 alpha in cancer cell invasiveness. Nat Cell Biol 6, 507-514.
13. Gaffney, E. V. (1982). A cell line (HBL-100) established from human breast milk. Cell Tissue Res 227, 563-568.
14. Gasc, J. M., Renoir, J. M., Radanyi, C., Joab, I., Tuohimaa, P., and Baulieu, E. E. (1984). Progesterone receptor in the chick oviduct: an immunohistochemical study with antibodies to distinct receptor components. J Cell Biol 99, 1193-1201.
15. Gopal U, Bohonowych J E, Lema-Tome C, Liu A, Garrett-Mayer E, Wang B. and Isaacs JS. (2011) A novel extracellular Hsp90 mediated co-receptor function for LRP1 regulates EphA2 dependent glioblastoma cell invasion. PLoS One. 8, e17649.
16. Grenert, J. P., Sullivan, W. P., Fadden, P., Haystead, T. A., Clark, J., Mimnaugh, E., Krutzsch, H., Ochel, H. J., Schulte, T. W., Sausville, E., Neckers, L. M., and Toft, D. O. (1997). The amino-terminal domain of heat shock protein 90 (hsp90) that binds geldanamycin is an ATP/ADP switch domain that regulates hsp90 conformation. J Biol Chem 272, 23843-23850.
17. Herz, J., and Strickland, D. K. (2001). LRP: a multifunctional scavenger and signaling receptor. J Clin Invest 108, 779-784.
18. Hutchison, G. J., Valentine, H. R., Loncaster, J. A., Davidson, S. E., Hunter, R. D., Roberts, S. A., Harris, A. L., Stratford, I. J., Price, P. M., and West, C. M. (2004). Hypoxia-inducible factor 1 alpha expression as an intrinsic marker of hypoxia: correlation with tumor oxygen, pimonidazole measurements, and outcome in locally advanced carcinoma of the cervix. Clin Cancer Res 10, 8405-8412.
19. Kamal, A., Thao, L., Sensintaffar, J., Zhang, L., Boehm, M. F., Fritz, L. C., and Burrows, F. J. (2003). A high-affinity conformation of Hsp90 confers tumour selectivity on Hsp90 inhibitors. Nature 425, 407-410.
20. Li, W., Henry, G., Fan, J., Bandyopadhyay, B., Pang, K., Garner, W., Chen, M., and Woodley, D. T. (2004). Signals that initiate, augment, and provide directionality for human keratinocyte motility. J Invest Dermatol 123, 622-633.
21. Li, W., Li, Y., Guan, S., Fan, J., Cheng, C. F., Bright, A. M., Chinn, C., Chen, M., and Woodley, D. T. (2007). Extracellular heat shock protein-90alpha: linking hypoxia to skin cell motility and wound healing. EMBO J. 26, 1221-1233.
22. Lundgren, K., Holm, C., and Landberg, G. (2007). Hypoxia and breast cancer: prognostic and therapeutic implications. Cell Mol Life Sci 64, 3233-3247.
23. Majmundar, A. J., Wong, W. J., and Simon, M. C. (2010). Hypoxia-inducible factors and the response to hypoxic stress. Mol Cell 40, 294-309.
24. Milani, M., and Harris, A. L. (2008). Targeting tumour hypoxia in breast cancer. Eur J Cancer 44, 2766-2773.
25. Neckers, L., and Neckers, K. (2002). Heat-shock protein 90 inhibitors as novel cancer chemotherapeutic agents. Expert Opin Emerg Drugs 7, 277-288.
26. Poon, E., Harris, A. L., and Ashcroft, M. (2009). Targeting the hypoxia-inducible factor (HIF) pathway in cancer. Expert Rev Mol Med 11, e26.
27. Rosman, D. S., Kaklamani, V., and Pasche, B. (2007). New insights into breast cancer genetics and impact on patient management. Curr Treat Options Oncol 8, 61-73.
28. Semenza, G. L. (2003). Targeting HIF-1 for cancer therapy. Nat Rev Cancer 3, 721-732.
29. Semenza, G. L. (2007). Life with oxygen. Science 318, 62-64.
30. Sheehan, T. J., Gershman, S. T., MacDougall, L. A., Danley, R. A., Mroszczyk, M., Sorensen, A. M., and Kulldorff, M. (2000). Geographic assessment of breast cancer screening by towns, zip codes, and census tracts. J Public Health Manag Pract 6, 48-57.
31. Sidera, K., Samiotaki, M., Yfanti, E., Panayotou, G., and Patsavoudi, E. (2004). Involvement of cell surface HSP90 in cell migration reveals a novel role in the developing nervous system. J Biol Chem 279, 45379-45388.
32. Sidera K, Gaitanou M, Stellas D, Matsas R. and Patsavoudi E. (2008) A critical role for HSP90 in cancer cell invasion involves interaction with the extracellular domain of HER-2. J Biol. Chem. 283, 2031-2041.
33. Solit, D. B., and Chiosis, G. (2008). Development and application of Hsp90 inhibitors. Drug Discov Today 13, 38-43.
34. Song, H., Li, Y., Lee, J., Schwartz, A. L., and Bu, G. (2009). Low-density lipoprotein receptor-related protein 1 promotes cancer cell migration and invasion by inducing the expression of matrix metalloproteinases 2 and 9. Cancer Res 69, 879-886.
35. Stellas D, El Hamidieh A, Patsavoudi E. (2010) Monoclonal antibody 4C5 prevents activation of MMP2 and MMP9 by disrupting their interaction with extracellular HSP90 and inhibits formation of metastatic breast cancer cell deposits. BMC Cell Biol. 2010 Jul. 5; 11:51.
36. Supko, J. G., Hickman, R. L., Greyer, M. R., and Malspeis, L. (1995). Preclinical pharmacologic evaluation of geldanamycin as an antitumor agent. Cancer Chemother Pharmacol 36, 305-315.
37. Trepel, J., Mollapour, M., Giaccone, G., and Neckers, L. (2010). Targeting the dynamic HSP90 complex in cancer. Nat Rev Cancer 10, 537-549.
38. Tsutsumi, S., and Neckers, L. (2007). Extracellular heat shock protein 90: a role for a molecular chaperone in cell motility and cancer metastasis. Cancer Sci 98, 1536-1539.
39. Tsutsumi, S., Scroggins, B., Koga, F., Lee, M. J., Trepel, J., Felts, S., Carreras, C., and Neckers, L. (2008). A small molecule cell-impermeant Hsp90 antagonist inhibits tumor cell motility and invasion. Oncogene 27, 2478-2487.
40. Welch, W. J., and Feramisco, J. R. (1982). Purification of the major mammalian heat shock proteins. J Biol Chem 257, 14949-14959.
41. Whitesell, L., Mimnaugh, E. G., De Costa, B., Myers, C. E., and Neckers, L. M. (1994). Inhibition of heat shock protein HSP90-pp 60v-src heteroprotein complex formation by benzoquinone ansamycins: essential role for stress proteins in oncogenic transformation. Proc Natl Acad Sci USA 91, 8324-8328.
42. Woodley, D. T., Fan, J., Cheng, C. F., Li, Y., Chen, M., Bu, G., and Li, W. (2009). Participation of the lipoprotein receptor LRP1 in hypoxia-HSP90alpha autocrine signaling to promote keratinocyte migration. J Cell Sci 122, 1495-1498.
43. Zhou, L., Yan, C., Gieling, R. G., Kida, Y., Garner, W., Li, W., and Han, Y. P. (2009). Tumor necrosis factor-alpha induced expression of matrix metalloproteinase-9 through p21-activated kinase-1. BMC Immunol 10,15.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. An isolated peptide comprising the sequence set forth in SEQ ID NO: 3, SEQ ID NO: 4, SEQ ID NO: 5 or SEQ ID NO: 6.

2. A nucleic acid molecule encoding the peptide of claim 1.

3. A cDNA molecule of claim 2.

4. A vector comprising the cDNA molecule of claim 3.

5. A host-vector system comprising the vector of claim 4 transfected into a compatible host cell.

6. The host-vector system of claim 5, wherein the compatible host cell is a prokaryotic cell or a eukaryotic cell.

7. An antibody which recognizes and binds an epitope comprising the sequence set forth in SEQ ID NO: 3, SEQ ID NO: 4, SEQ ID NO: 5 and/or SEQ ID NO: 6.

8. The antibody of claim 7, wherein the antibody is a monoclonal antibody or fragment thereof, a polyclonal antibody or a fragment thereof, chimeric antibodies, humanized antibodies, human antibodies, or a single chain antibody.

9. A pharmaceutical composition comprising the antibody of claim 7 and a pharmaceutical carrier.

10. A vaccine comprising the isolated peptide of claim 1.

11. A method for treating HIF-1α-overexpressing cancer in a subject in need thereof comprising:
(a) providing a composition comprising an inhibitor of Hsp90α; and
(b) administering a therapeutically effective amount of the composition to the subject so as to treat HIF-1-positive cancer in the subject.

12. (canceled)

13. (canceled)

14. The method of claim 11, wherein the inhibitor of Hsp90α targets the amino acids of Hsp90α as set forth in SEQ ID NO: 3, SEQ ID NO: 4,
SEQ ID NO: 5 and/or SEQ ID NO:6.

15. The method of claim 11, wherein the inhibitor of Hsp90α is selected from the group consisting of a small molecule, a peptide, an antibody or a fragment thereof and a nucleic acid molecule.

16. The method of claim 11, wherein the inhibitor of Hsp90α is an inhibitor of Hsp90α eceptor and is selected from the group consisting of a small molecule, a peptide, an antibody or a fragment thereof or a nucleic acid molecule.

17. The method of claim 15, wherein the nucleic acid molecule is a siRNA molecule.

18. The method of claim 15, wherein the antibody is selected from the group consisting of monoclonal antibody or fragment thereof, a polyclonal antibody or a fragment thereof, chimeric antibodies, humanized antibodies, human antibodies, and a single chain antibody.

19-29. (canceled)


SEQUENCE LISTING (6 sequences)

SEQ ID NO: 1 - DNA, 2220 bp, Homo sapiens
  CDS (1)..(2199)
atgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaagcttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaacatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgactt

SEQ ID NO: 2 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 3 - DNA, 345 bp, Homo sapiens
  CDS (1)..(345)
gaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgat

SEQ ID NO: 4 - protein, 115 aa, Homo sapiens
EEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFD

SEQ ID NO: 5 - DNA, 81 bp, Homo sapiens
  CDS (1)..(81)
tctgatgaggaagaagaaaagaaggatggtgacaagaagaagaagaagaagattaaggaaaagtacatcgatcaagaagag

SEQ ID NO: 6 - protein, 27 aa, Homo sapiens
SDEEEEKKDGDKKKKKKIKEKYIDQEE